SEQUENCE LISTING (4 sequences)

SEQ ID NO: 1 - protein, 322 aa, Homo sapiens [sequence omitted]

SEQ ID NO: 2 - DNA, 969 bp, Homo sapiens
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 3 - protein, 329 aa, Artifical Sequence
  misc_feature: Chimeric AA sequence of human arginase I and an N-terminal histidine tag
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 4 - DNA, 990 bp, Artifical Sequence
  misc_feature: Chimeric DNA sequence encoding human arginase I and an N-terminal histidine tag
atgcatcaccatcaccatcacatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

(19) United States
(12) Patent Application Publication
(10) Pub. No.: US 2018/0271960 A1
(43) Pub. Date: Sep. 27, 2018

(54) METHOD TO TREAT CANCER USING ARGININE DELPETOR AND ORNITHINE DECARBOXYLASE (ODC) INHIBITOR

(71) Applicant: Bio-Cancer Treatment International Ltd., Hong Kong (CN)

(72) Inventors: Ning Man CHENG, Hong Kong (CN); Kin Pong U, Hong Kong (CN); Sze Kwan LAM, Hong Kong (CN); Chung Man HO, Hong Kong (CN)

(21) Appl. No.: 15/761,429

(22) PCT Filed: Sep. 21, 2016
(86) PCT No.: PCT/CN2016/099642

§ 371 Date: Mar. 20, 2018

Related U.S. Application Data
(60) Provisional application No. 62/221,604, filed on Sep. 21, 2015.

(51) Int. Cl. A61K 38/50; A61K 47/60; A61K 31/198; A61P 35/00

(52) U.S. Cl. CPC A61K 38/50; A61K 47/60; A61P 35/00; A61K 31/198; C12Y 305/03001

(57) ABSTRACT

One example embodiment is a method of treating lung carcinoma in a subject in need thereof. The method includes administering to the subject a therapeutically effective amount of an arginine reducing compound and a therapeutically effective amount of an ornithine decarboxylase (ODC) inhibitor to provide a combination therapy that has a synergistic therapeutic effect compared to an effect of the arginine reducing compound and an effect of the ODC inhibitor, in which each of the arginine reducing compound and the ODC inhibitor is administered alone.

### FIELD OF THE INVENTION

The present invention relates to a method to treat cancer using an arginine depletor and an ornithine decarboxylase (ODC) inhibitor.

### REFERENCE TO SEQUENCE LISTING

The hard copy of the sequence listing submitted herewith and the corresponding computer readable form are both incorporated herein by reference in their entireties.

### BACKGROUND

Lung cancer is one of the most lethal cancers worldwide. Different drugs have been developed to treat lung cancer and some of the anti-cancer drugs may not be readily useful as a remedy to lung cancers.

In view of the demand for effectively treating lung cancers, improvements in method and compositions that treat lung cancers are desired.

### BRIEF DESCRIPTION OF THE DRAWINGS

FIG. 1 shows an expression of ornithine transcarbamylase (OTC) and argininosuccinate synthase (ASS1) in the tested lung adenocarcinomacell lines by Western blotin accordance with an example embodiment.

FIG. 2 shows a graph of ASS expression intensity in the tested lung adenocarcinomacell lines by Western blot in accordance with an example embodiment.

FIG. 3 shows an OTC expression characterization in the tested lung adenocarcinoma cell lines in accordance with an example embodiment.

FIG. 4 shows an ASS1 expression characterization in the tested lung adenocarcinoma cell lines in accordance with an example embodiment.

FIG. 5A shows a graph of a study of effects of PEG-BCT-100 and arginine deiminase (ADI) on H23 lung adenocarcinoma cell line in accordance with an example embodiment.

FIG. 5B shows a graph of a study of effects of PEG-BCT-100 and ADI on H358 lung adenocarcinoma cell line in accordance with an example embodiment.

FIG. 5C shows a graph of a study of effects of PEG-BCT-100 and ADI on HCC827 lung adenocarcinoma cell line in accordance with an example embodiment.

FIG. 5D shows a graph of a study of effects of PEG-BCT-100 and ADI on H1650 lung adenocarcinoma cell line in accordance with an example embodiment.

FIG. 5E shows a graph of a study of effects of PEG-BCT-100 and ADI on H1975 lung adenocarcinoma cell line in accordance with an example embodiment.

FIG. 5F shows a graph of a study of effects of PEG-BCT-100 and ADI on HCC2935 lung adenocarcinoma cell line in accordance with an example embodiment.

FIG. 5G shows a graph of a study of effects of PEG-BCT-100 and ADI on HCC4006 lung adenocarcinoma cell line in accordance with an example embodiment.

FIG. 5H shows a graph of a study of effects of PEG-BCT-100 and ADI on A549 lung adenocarcinoma cell line in accordance with an example embodiment.

FIG. 6A shows a graph of a study of growth inhibition effect of arginine deiminase (ADI) on H23 lung adenocarcinoma cell line in accordance with an example embodiment.

FIG. 6B shows a graph of a study of growth inhibition effect by PEG-BCT-100 on H23 lung adenocarcinoma cell line in accordance with an example embodiment.

FIG. 7A shows a graph of a study of growth inhibition effect by arginine deiminase (ADI) on H358 lung adenocarcinoma cell line in accordance with an example embodiment.

FIG. 7B shows a graph of a study of growth inhibition effect by PEG-BCT-100 on H358 lung adenocarcinoma cell line in accordance with an example embodiment.

FIG. 8A shows a graph of a study of growth inhibition effect by arginine deiminase (ADI) on HCC827 lung adenocarcinoma cell line in accordance with an example embodiment.

FIG. 8B shows a graph of a study of growth inhibition effect by PEG-BCT-100 on HCC827 lung adenocarcinoma cell line in accordance with an example embodiment.

FIG. 9A shows a graph of a study of growth inhibition effect by arginine deiminase (ADI) on H1650 lung adenocarcinoma cell line in accordance with an example embodiment.

FIG. 9B shows a graph of a study of growth inhibition effect by PEG-BCT-100 on H1650 lung adenocarcinoma cell line in accordance with an example embodiment.

FIG. 10A shows a graph of a study of growth inhibition effect by arginine deiminase (ADI) on H1975 lung adenocarcinoma cell line in accordance with an example embodiment.

FIG. 10B shows a graph of a study of growth inhibition effect by PEG-BCT-100 on H1975 lung adenocarcinoma cell line in accordance with an example embodiment.

FIG. 11A shows a graph of a study of growth inhibition effect by arginine deiminase (ADI) on HCC2935 lung adenocarcinoma cell line in accordance with an example embodiment.

FIG. 11B shows a graph of a study of growth inhibition effect by PEG-BCT-100 on HCC2935 lung adenocarcinoma cell line in accordance with an example embodiment.

FIG. 12A shows a graph of a study of growth inhibition effect by arginine deiminase (ADI) on HCC4006 lung adenocarcinoma cell line in accordance with an example embodiment.

FIG. 12B shows a graph of a study of growth inhibition effect by PEG-BCT-100 on HCC4006 lung adenocarcinoma cell line in accordance with an example embodiment.

FIG. 13A shows a graph of a study of growth inhibition effect by arginine deiminase (ADI) on A549 lung adenocarcinoma cell line in accordance with an example embodiment.

FIG. 13B shows a graph of a study of growth inhibition effect by PEG-BCT-100 on A549 lung adenocarcinoma cell line in accordance with an example embodiment.

FIG. 14 shows a table of average IC₅₀ values for PEG-BCT-100 and ADI on the tested lung adenocarcinoma cell lines in accordance with an example embodiment.

FIG. 15A shows a panel of pictures on internalization of PEG-BCT-100 by H358 lung adenocarcinoma cells assessed by immunocytochemistry in accordance with an example embodiment.

FIG. 15B shows a graph of normalized arginine concentration against dosage on H358 lung adenocarcinoma cells in accordance with an example embodiment.

FIG. 15C shows a panel of pictures on internalization of PEG-BCT-100 by H1650 lung adenocarcinoma cells assessed by immunocytochemistry in accordance with an example embodiment.

FIG. 15D shows a graph of normalized arginine concentration against dosage on H1650 lung adenocarcinoma cells in accordance with an example embodiment.

FIG. 15E shows a panel of pictures on internalization of PEG-BCT-100 by H1975 lung adenocarcinoma cells assessed by immunocytochemistry in accordance with an example embodiment.

FIG. 15F shows a graph of normalized arginine concentration against dosage on H1975 lung adenocarcinoma cells in accordance with an example embodiment.

FIG. 15G shows a panel of pictures on internalization of PEG-BCT-100 by HCC4006 lung adenocarcinoma cells assessed by immunocytochemistry in accordance with an example embodiment.

FIG. 15H shows a graph of normalized arginine concentration against dosage on HCC4006 lung adenocarcinoma cells in accordance with an example embodiment.

FIG. 16A shows a graph of effects of PEG-BCT-100 on H1650 lung adenocarcinoma xenografts in BALB/c nude mice in accordance with an example embodiment.

FIG. 16B shows a graph of effects of PEG-BCT-100 on H1975 lung adenocarcinoma xenografts in BALB/c nude mice in accordance with an example embodiment.

FIG. 16C shows a graph of effects of PEG-BCT-100 on HCC4006 lung adenocarcinoma xenografts in BALB/c nude mice in accordance with an example embodiment.

FIG. 17A shows a graph of in vivo ASS1 expression in H1650 xenograft in control arm and PEG-BCT-100 treatment arm in accordance with an example embodiment.

FIG. 17B shows a graph of in vivo ornithine decarboxylase (ODC) expression in H1650 xenograft in control arm and PEG-BCT-100 treatment arm in accordance with an example embodiment.

FIG. 17C shows a panel of in vivo expressions of ASS1, OTC and ODC in H1650 xenograft in control arm and PEG-BCT-100 treatment arm by Western blot in accordance with an example embodiment.

FIG. 17D shows a graph of in vivo ASS1 expression in H1975 xenograft in control arm and PEG-BCT-100 treatment arm in accordance with an example embodiment.

FIG. 17E shows a graph of in vivo ODC expression in H1975 xenograft in control arm and PEG-BCT-100 treatment arm in accordance with an example embodiment.

FIG. 17F shows a panel of in vivo expressions of ASS1, OTC and ODC in H1975 xenograft in control arm and PEG-BCT-100 treatment arm by Western blot in accordance with an example embodiment.

FIG. 17G shows a graph of in vivo ASS1 expression in HCC4006 xenograft in control arm and PEG-BCT-100 treatment arm in accordance with an example embodiment.

FIG. 17H shows a panel of in vivo expressions of ASS1, OTC and ODC in HCC4006 xenograft in control arm and PEG-BCT-100 treatment arm by Western blot in accordance with an example embodiment.

FIG. 18 shows a graph of in vivo effects of PEG-BCT-100 and/or DFMO in H1650 lung adenocarcinoma xenografts in accordance with an example embodiment.

FIG. 19A shows a graph of median survival of BALB/c nude mice with H1650 lung adenocarcinoma xenografts upon PEG-BCT-100 and/or DFMO treatments in accordance with an example embodiment.

FIG. 19B shows a table of median survival of BALB/c nude mice with H1650 lung adenocarcinoma xenografts upon PEG-BCT-100 and/or DFMO treatments in accordance with an example embodiment.

FIG. 20 shows a graph of in vivo effects of PEG-BCT-100 and/or DFMO in H1975 lung adenocarcinoma xenografts in accordance with an example embodiment.

FIG. 21A shows a graph of median survival of BALB/c nude mice with H1975 lung adenocarcinoma xenografts upon PEG-BCT-100 and/or DFMO treatments in accordance with an example embodiment.

FIG. 21B shows a table of median survival of BALB/c nude mice with H1975 lung adenocarcinoma xenografts upon PEG-BCT-100 and/or DFMO treatments in accordance with an example embodiment.

FIG. 22 shows a graph of in vivo effects of PEG-BCT-100 and/or DFMO in HCC4006 lung adenocarcinoma xenografts in accordance with an example embodiment.

FIG. 23A shows a graph of in vivo effects of PEG-BCT-100 and/or DFMO in SU-DHL-6 B cell lymphoma xenograft model in accordance with an example embodiment.

FIG. 23B shows a table of anti-tumour activity of PEG-BCT-100 and/or DFMO in SU-DHL-6 B cell lymphoma xenograft model in accordance with an example embodiment.

FIG. 24A shows a graph of in vivo effects of PEG-BCT-100 and/or DFMO in SU-DHL-10 B cell lymphoma xenograft model in accordance with an example embodiment.

FIG. 24B shows a table of anti-tumour activity of PEG-BCT-100 and/or DFMO in SU-DHL-10 B cell lymphoma xenograft model in accordance with an example embodiment.

FIG. 25A shows a graph of in vivo effects of PEG-BCT-100 and/or DFMO in Kasumi-1 leukemia xenograft model in accordance with an example embodiment.

FIG. 25B shows a table of anti-tumour activity of PEG-BCT-100 and/or DFMO in Kasumi-1 leukemia xenograft model in accordance with an example embodiment.

FIG. 26A shows a graph of in vivo effects of PEG-BCT-100 and/or DFMO in K-562 leukemia xenograft model in accordance with an example embodiment.

FIG. 26B shows a table of anti-tumour activity of PEG-BCT-100 and/or DFMO in K-562 leukemia xenograft model in accordance with an example embodiment.

FIG. 27A shows a graph of in vivo effects of PEG-BCT-100 and/or DFMO in Hep G2 hepatocellular carcinoma xenograft model in accordance with an example embodiment.

FIG. 27B shows a table of anti-tumour activity of PEG-BCT-100 and/or DFMO in Hep G2 hepatocellular carcinoma xenograft model in accordance with an example embodiment.

FIG. 28A shows a graph of in vivo effects of PEG-BCT-100 and/or DFMO in Hep 3B hepatocellular carcinoma xenograft model in accordance with an example embodiment.

FIG. 28B shows a table of anti-tumour activity of PEG-BCT-100 and/or DFMO in Hep 3B hepatocellular carcinoma xenograft model in accordance with an example embodiment.

FIG. 29A shows a graph of in vivo effects of PEG-BCT-100 and/or DFMO in MIA-PaCa-2 pancreatic cancer xenograft model in accordance with an example embodiment.

FIG. 29B shows a table of anti-tumour activity of PEG-BCT-100 and/or DFMO in MIA-PaCa-2 pancreatic cancer xenograft model in accordance with an example embodiment.

FIG. 30A shows a graph of in vivo effects of PEG-BCT-100 and/or DFMO in CAPAN-1 pancreatic cancer xenograft model in accordance with an example embodiment.

FIG. 30B shows a table of anti-tumour activity of PEG-BCT-100 and/or DFMO in CAPAN-1 pancreatic cancer xenograft model in accordance with an example embodiment.

### SUMMARY OF THE INVENTION

One example embodiment is a method of treating lung carcinoma in a subject in need thereof. The method includes administering to the subject a therapeutically effective amount of an arginine reducing compound and a therapeutically effective amount of an ornithine decarboxylase (ODC) inhibitor to provide a combination therapy that has a synergistic therapeutic effect compared to an effect of the arginine reducing compound and an effect of the ODC inhibitor, in which each of the arginine reducing compound and the ODC inhibitor is administered alone.

Other example embodiments are discussed herein.

### DETAILED DESCRIPTION

Example embodiments relate to methods and pharmaceutical composition that treat lung cancers.

Arginine, a semi-essential amino acid, is involved in many metabolic processes and is also important for growth of some cancer cells. Arginine depletion plays a useful role in the treatment of some cancers, but may not be proficient in treating other types of cancer. In fact, administration of arginase may even cause proliferation in some tumor cells.

The present inventors have determined that when arginase converts arginine into ornithine, certain types of cancer cells would upregulate ornithine decarboxylase (ODC). ODC then converts ornithine into polyamines, increasing the capability of these cancer cells to proliferate, invade and metastasize to new tissues.

The present inventors have further determined that even in ODC negative cells, the administration of arginase (and hence resulting in the increase of ornithine) may result in upregulation of ODC, inducing them to become ODC positive in certain cancer cells to result in increased polyamines.

In one example embodiment, the lung carcinoma is lung adenocarcinoma. In another example embodiment, the arginine reducing compound is a pegylated recombinant human arginase. By way of example, the recombinant human arginase has an amino acid sequence of SEQ ID NO:1.

In another example embodiment, the ODC inhibitor is difluoromethylornithine (DFMO). In yet another example embodiment, the arginine reducing compound and the ODC inhibitor are administered concurrently.

In an example embodiment, the cancer cells of the lung carcinoma are ODC positive. In one example embodiment, the cancer cells of the lung carcinoma are ODC negative. In another example embodiment, the cancer cells of the lung carcinoma are argininosuccinate synthase negative (ASS1⁻) or ornithine transcarbamylase negative (OTC⁻).

One example embodiment is therefore to treat lung cancer by blocking ODC in cancer cells in addition to depleting arginine by an administration of arginine depleting compound. A therapeutically effective amount of the arginine depleting compound and a therapeutically effective amount of an ODC blocking agent are administered to the subject, where the administration provides a synergistic therapeutic effect compared to an effect in treating lung cancer of the arginine depleting compound and an effect in treating lung cancer of the ODC blocking agent, in which each of the arginine depleting compound and the ODC blocking agent is administered alone.

In one example embodiment, the lung cancer is lung adenocarcinoma.

In another example embodiment, the arginine depleting compound is a pegylated recombinant human arginase. By way of example, the recombinant human arginase has an amino acid sequence of SEQ ID NO:1.

In another example embodiment, the ODC blocking agent is DFMO.

In an example embodiment, these cancer cells may be either ODC negative or ODC positive. In another example embodiment, these cancer cells are argininosuccinate synthase negative or ornithine transcarbamylase negative.

One example embodiment relates to a method for inhibiting proliferation of cancer cells of lung adenocarcinoma. The method includes contacting the cancer cells with an arginine depleting compound in combination with an ornithine decarboxylase (ODC) inhibitor. The combination provides a synergistic anti-cancerous effect compared to an effect of the arginine depleting compound and an effect of the ODC inhibitor, each administered alone

In an example embodiment, the arginine depleting compound is a pegylated recombinant human arginase. By way of example, the recombinant human arginase has an amino acid sequence of SEQ ID NO:1.

In another example embodiment, the ODC inhibitor is DFMO. In one example embodiment, the arginine reducing depleting compound and the ODC inhibitor are administered concurrently.

One example embodiment relates to a pharmaceutical composition for use in a synergistic treatment of lung cancer. The pharmaceutical composition includes an arginine depleting compound and an inhibitor of ODC.

In an example embodiment, the lung cancer is lung adenocarcinoma.

In another example embodiment, the arginine depleting compound is a pegylated recombinant human arginase. By way of example, the recombinant human arginase has an amino acid sequence of SEQ ID NO:1.

In one example embodiment, the inhibitor of ODC is DFMO.

In another example embodiment, an amount of the arginine depleting compound and an amount of the inhibitor of ODC are effective for therapy in a subject, and the subject is a human.

### Example 1

One example embodiment studies in vitro characterization of argininosuccinate synthase (ASS1) and ornithine transcarbamylase (OTC) expression in lung adenocarcinomas.

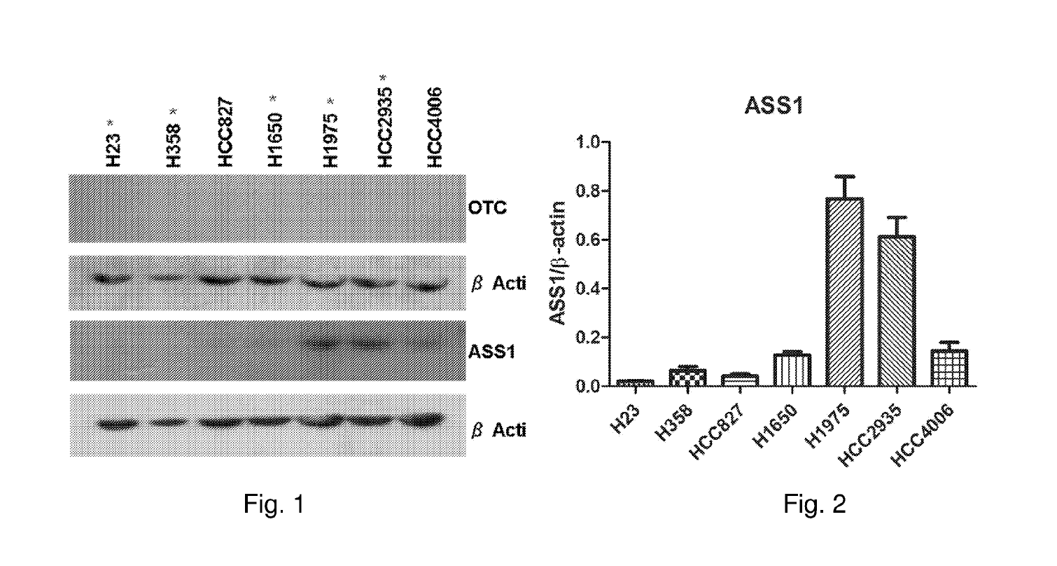

In one example embodiment, eight lung adenocarcinoma cell lines (i.e. H23, H358, HCC827, H1650, H1975, HCC2935, HCC4006, and A549), obtained from American Type Culture Collection (ATCC), are assessed for ornithine transcarbamylase (OTC) and argininosuccinate synthase (ASS1) protein expression by immunocytochemistry and Western blot. The level of expression is determined by normalizing against housekeeping protein (β-actin) in Western blot. Results of this study are presented in FIGS. 1-4.

FIGS. 1 and 2 show an expression of OTC and ASS1 in seven of the tested lung adenocarcinomacell lines (i.e. H23, H358, HCC827, H1650, H1975, HCC2935, and HCC4006\) respectively by Western blot and immunocytochemistry. All examined lung adenocarcinomas are either OTC negative (OTC⁻), ASS1 negative (ASS1⁻), or OTC⁻/ASS1⁻. Cell lines with an asterisk (*) are erlotinib resistant cell lines.

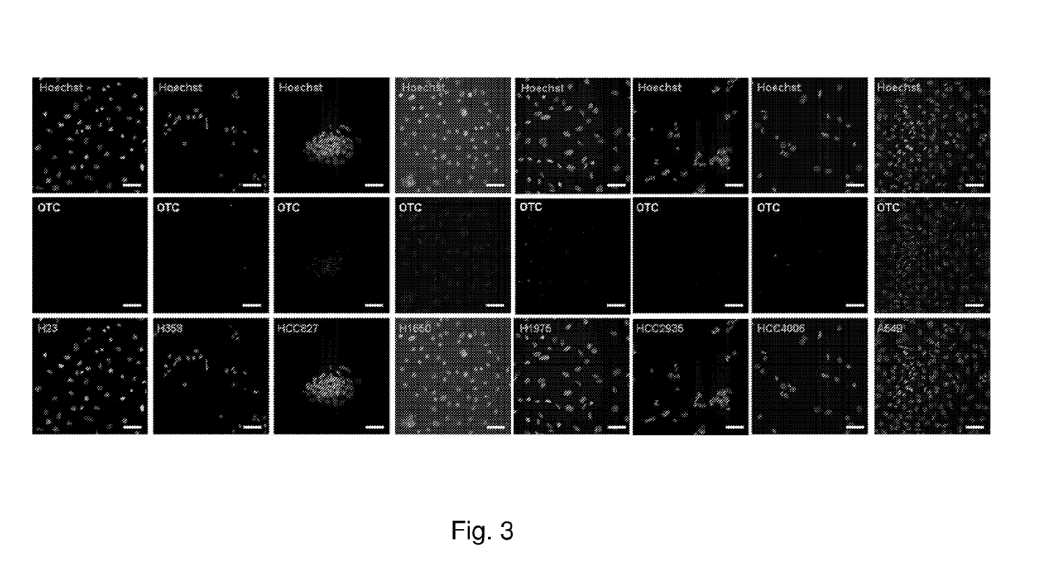

FIG. 3 shows an OTC expression profile in the tested lung adenocarcinoma cell lines. All cell lines except A549, which is a positive control for OTC expression, are negative for OTC expression. The nuclei of lung adenocarcinoma are stained with Hoechst staining as shown in the top row, while OTC expression is detected by anti-OTC antibody (Sigma Aldrich) and Alexa Fluor 488 goat anti-rabbit secondary antibody as shown in the middle row. The images are merged to visualize the cytoplasmic localization of OTC protein. Scale bar represents 100 μm.

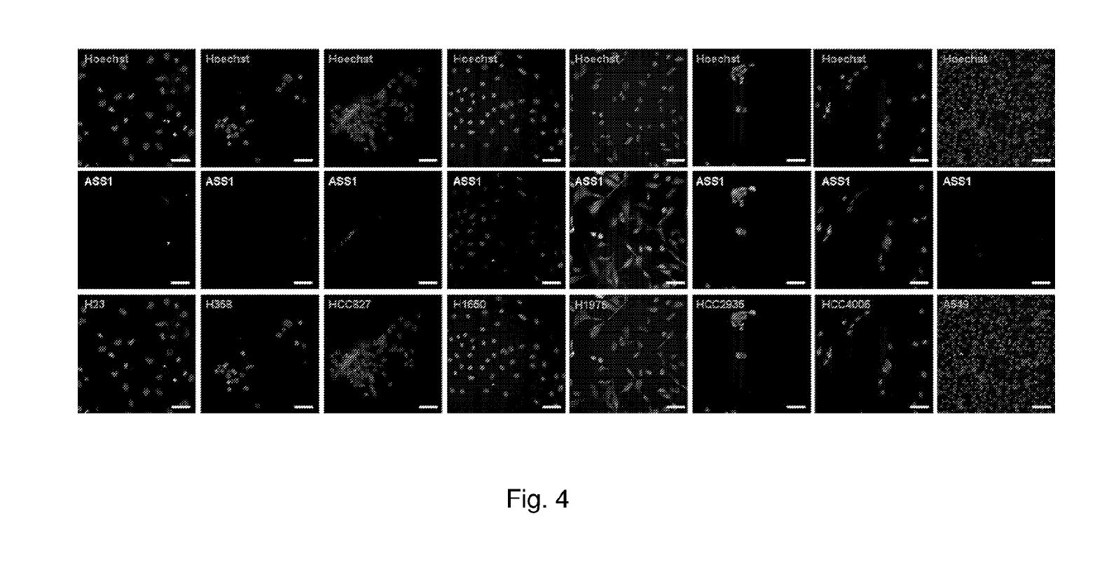

FIG. 4 shows an ASS1 expression characterization in the tested lung adenocarcinoma cell lines. Only H1650, H1975, HCC2935 and HCC4006 cells express ASS1. The nuclei of lung adenocarcinoma are stained with Hoechst staining as shown in the top row, while ASS1 expression is detected by anti-ASS1 antibody (Sigma Aldrich) and Alexa Fluor 488 goat anti-rabbit secondary antibody as shown in the middle row. The images are merged to visualize the cytoplasmic localization of ASS1 protein. Scale bar represents 100 μm.

As illustrated in FIGS. 1-4, all examined lung adenocarcinoma cell lines are either OTC⁻ or ASS1⁻ and so these cell lines are predicted to be sensitive to arginine depletion by PEG-BCT-100.

### Example 2

One example embodiment studies in vitro efficacy of PEG-BCT-100 and arginine deiminase (ADI) against lung adenocarcinoma.

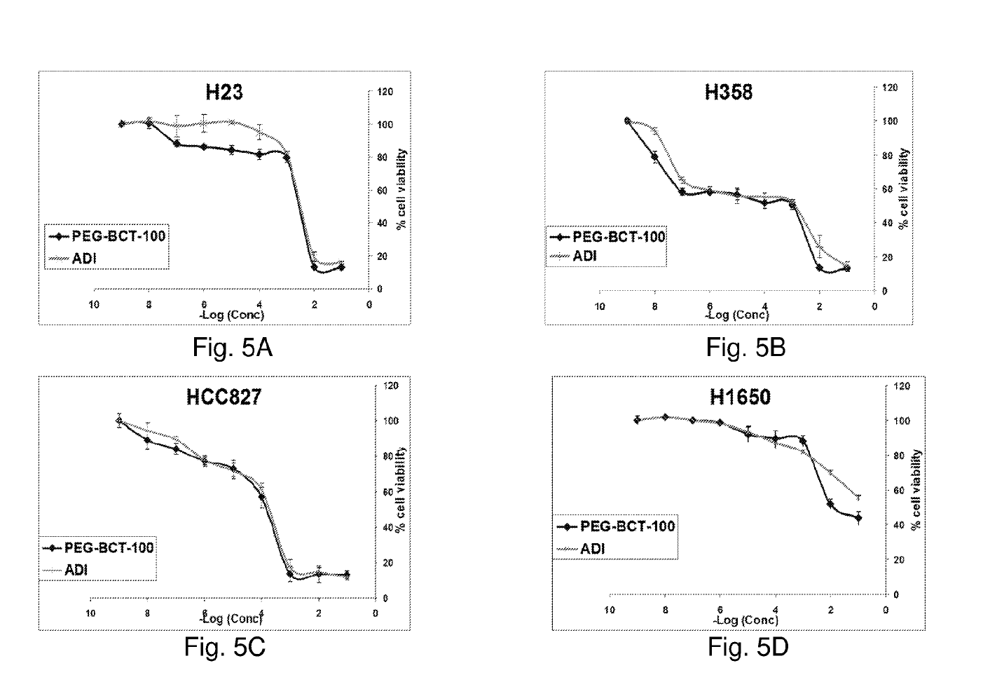

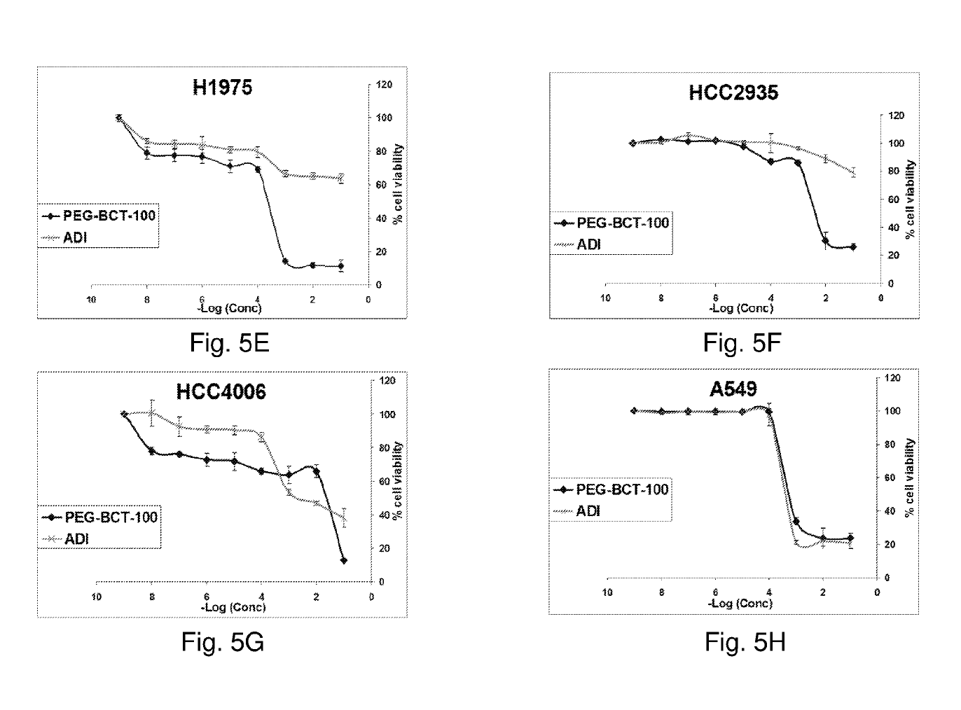

In one example embodiment, eight lung adenocarcinoma cell lines (i.e. H23, H358, HCC827, H1650, H1975, HCC2935, HCC4006, and A549), obtained from ATCC, are used to assess the in vitro efficacies of PEG-BCT-100 and ADI. The inhibition ratio (IR) and the half maximal inhibitory concentration (IC₅₀) values are determined by a MTT assay. Results of this study are presented in FIGS. 5A-14.

FIGS. 5A-5H show that PEG-BCT-100 induces cytotoxicity in all tested lung adenocarcinoma cell lines. ADI induces cytotoxicity in all cell lines but with less cytotoxic effects on cell lines H1650, H1975, HCC2935, and HCC4006. Cell viability is quantified by the MTT assay after treatment for 72 h.

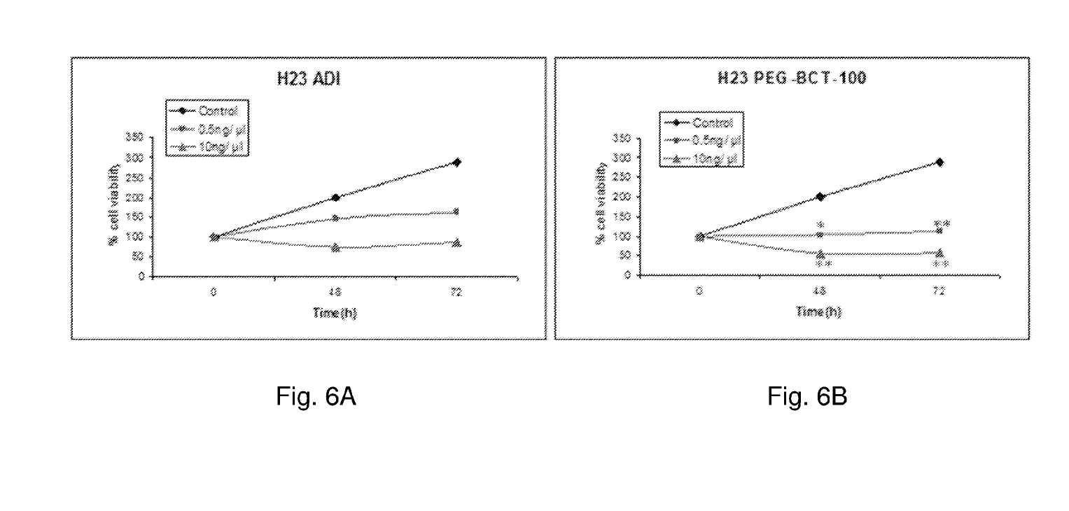

FIGS. 6A-6B show that PEG-BCT-100 inhibits H23 lung adenocarcinoma cell line proliferation more effectively than ADI over the course of 48 h and 72 h at 0.5 ng/μl and 10 ng/μl. (* p<0.05, ** p<0.01.)

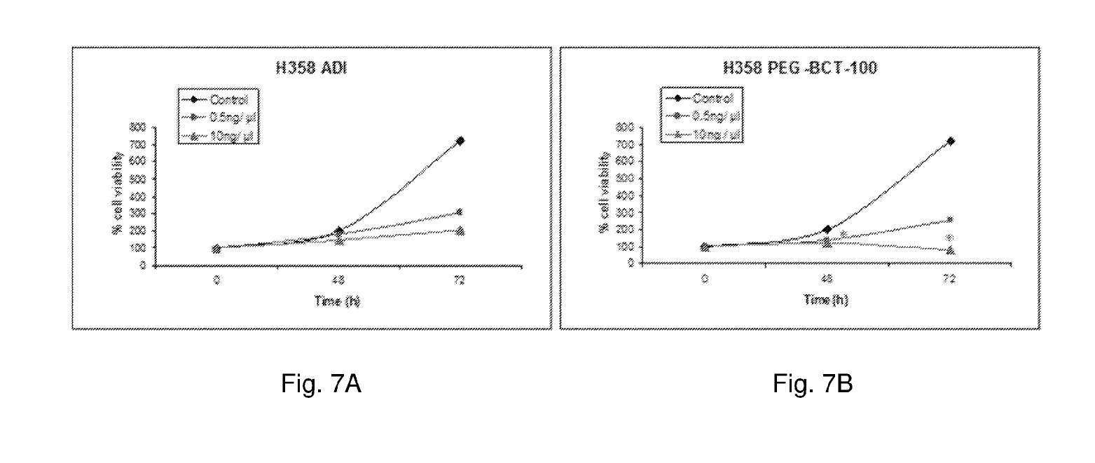

FIGS. 7A-7B show that PEG-BCT-100 inhibits H358 lung adenocarcinoma cell line proliferation more effectively than ADI over the course of 48 h and 72 h at 10 ng/μ1. (* p<0.05.)

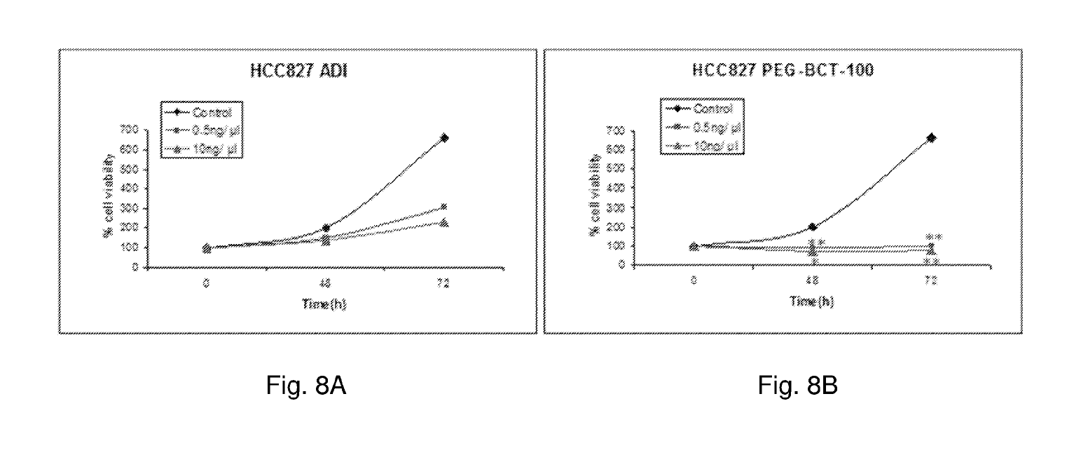

FIGS. 8A-8B show that PEG-BCT-100 inhibits HCC827 lung adenocarcinoma cell line proliferation more effectively than ADI over the course of 48 h and 72 h at 0.5 ng/μl and 10 ng/μl. (* p<0.05, ** p<0.01.)

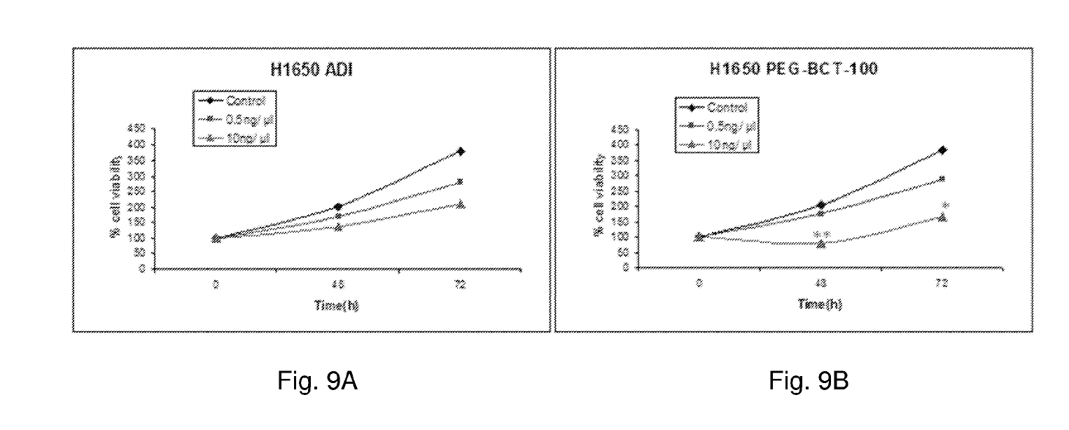

FIGS. 9A-9B show that PEG-BCT-100 inhibits H1650 lung adenocarcinoma cell line proliferation more effectively than ADI over the course of 48 h and 72 h at 10 ng/μ1. (* p<0.05, ** p<0.01.)

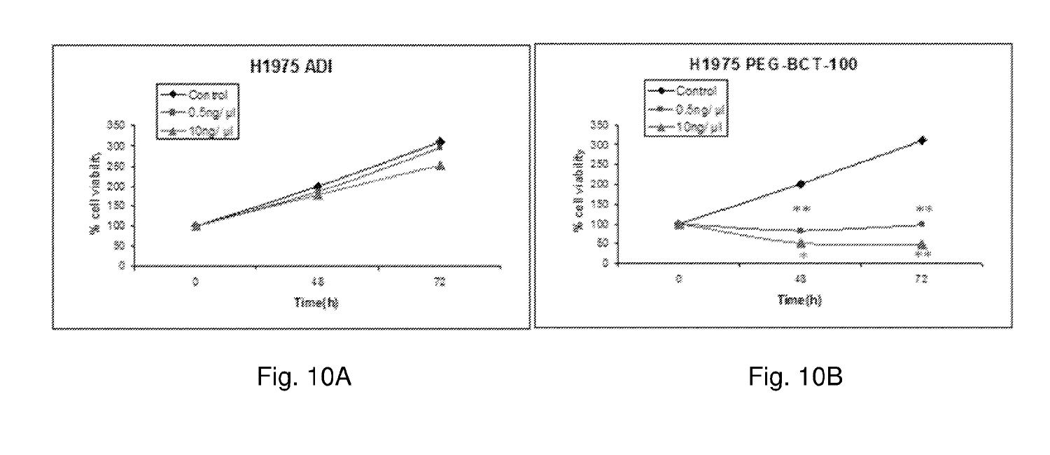

FIGS. 10A-10B show that PEG-BCT-100 inhibits H1975 lung adenocarcinoma cell line proliferation over the course of 48 h and 72 h at 0.5 ng/μl and 10 ng/μ1. ADI does not inhibit H1975 lung adenocarcinoma cell line proliferation. (* p<0.05, ** p<0.01).

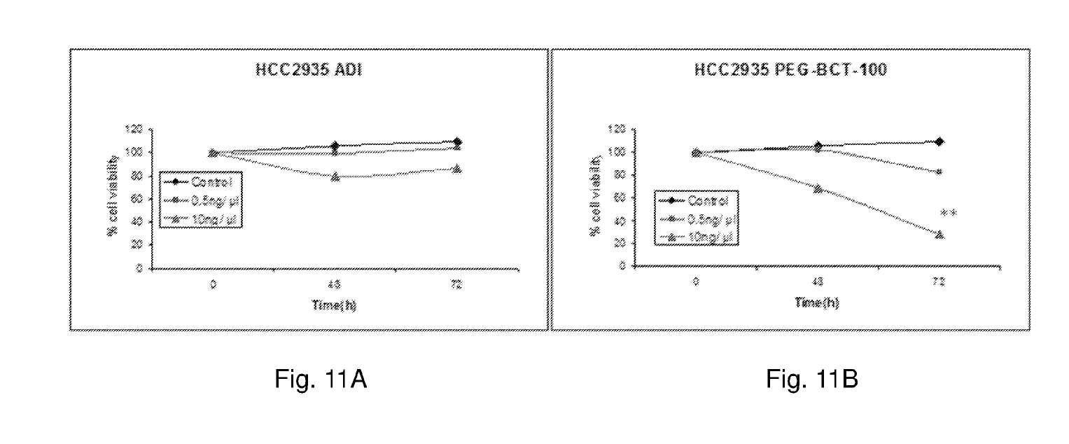

FIGS. 11A-11B show that PEG-BCT-100 induces toxicity in HCC2935 lung adenocarcinoma cell line over the course of 72 h at 10 ng/μl only. ADI does not inhibit HCC2935 lung adenocarcinoma cell line proliferation. (** p<0.01.)

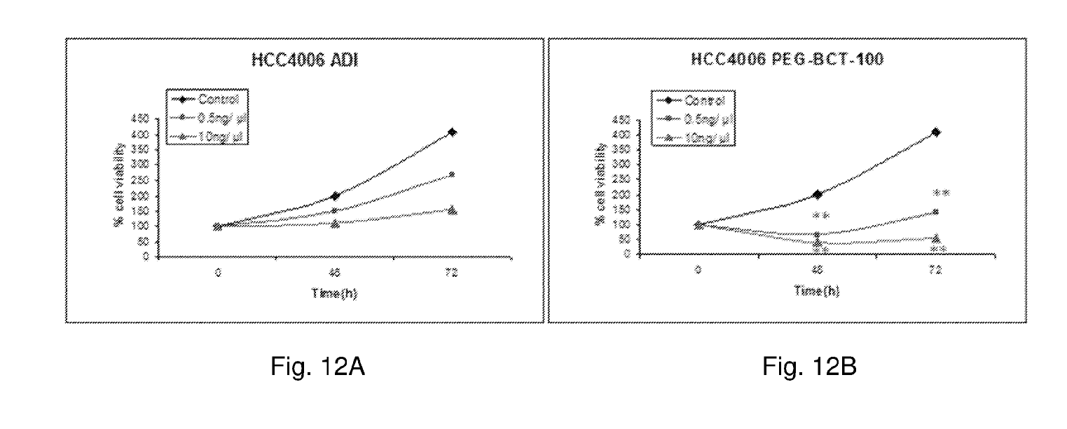

FIGS. 12A-12B show that PEG-BCT-100 inhibits HCC4006 lung adenocarcinoma cell line proliferation more effectively than ADI over the course of 48 h and 72 h at 0.5 ng/μl and 10 ng/μl. (** p<0.01.)

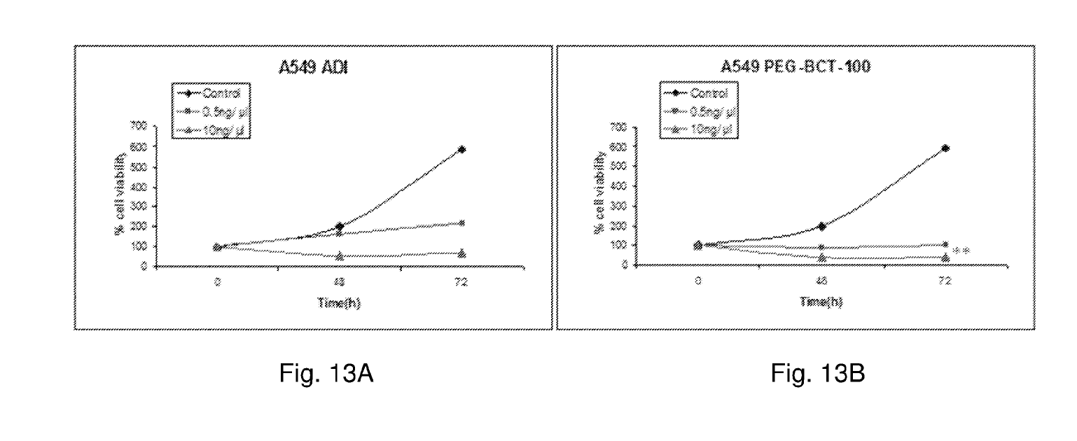

FIGS. 13A-13B show that PEG-BCT-100 inhibits A549 lung adenocarcinoma cell line proliferation more effectively than ADI over the course of 48 h and 72 h at 0.5 ng/μl and 10 ng/μl. (* p<0.05, ** p<0.01.)

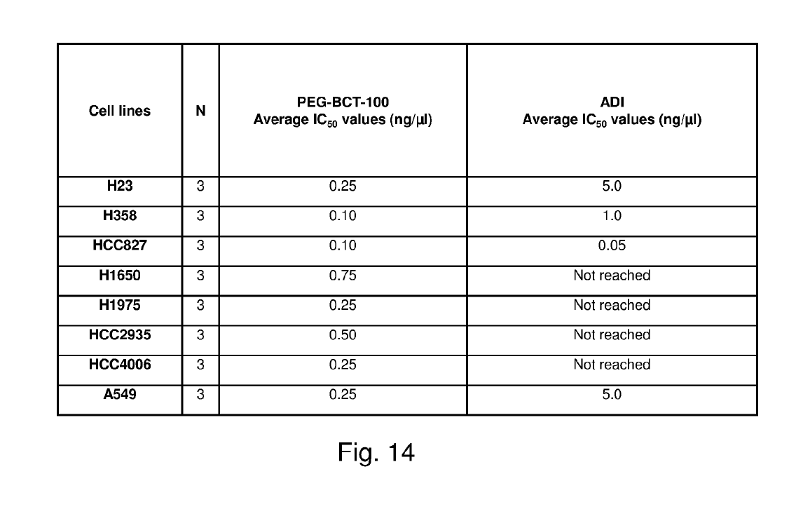

FIG. 14 shows average IC₅₀ values of the tested lung adenocarcinoma cell lines. As shown, PEG-BCT-100 is able to inhibit proliferation of all examined lung adenocarcinoma cell lines. On the other hand, ADI is less effective than PEG-BCT-100 in inhibiting cancer cell proliferation among ASS1⁻ cancer cell lines, while ASS1⁺ confers resistance to ADI treatment.

As illustrated in FIGS. 5A-14, all examined lung adenocarcinoma cell lines are either OTC⁻ or ASS1⁻ and so these cell lines are predicted to be sensitive to arginine depletion by PEG-BCT-100. Different lung adenocarcinoma cell lines display different sensitivities towards PEG-BCT-100. On the other hand, lung adenocarcinoma cell lines with ASS1 expression are resistant to ADI treatment.

### Example 3

One example embodiment studies in vitro efficacy of PEG-BCT-100 as an intracellular arginine-depleting agent.

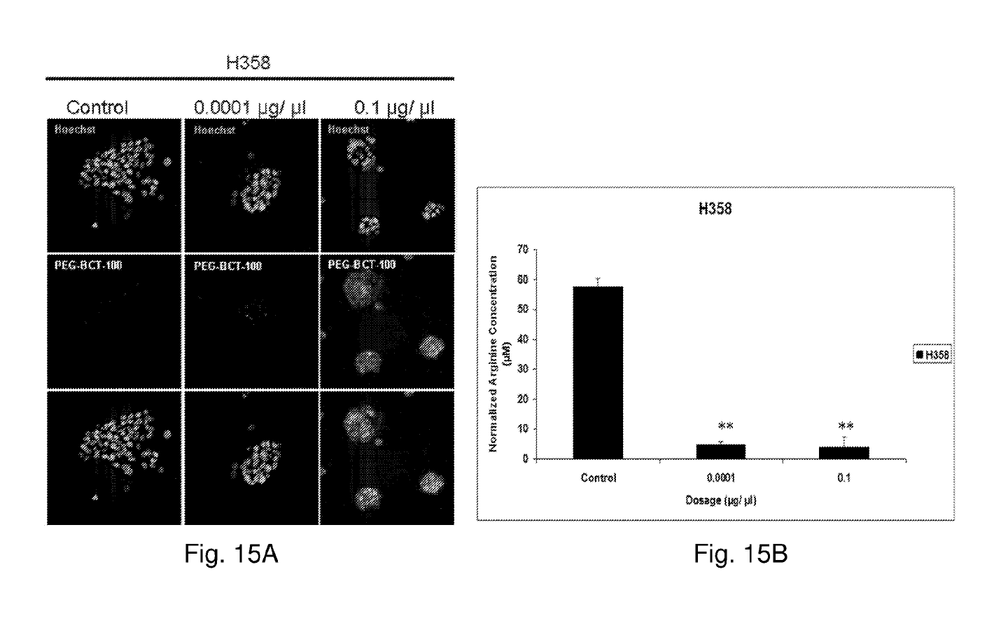

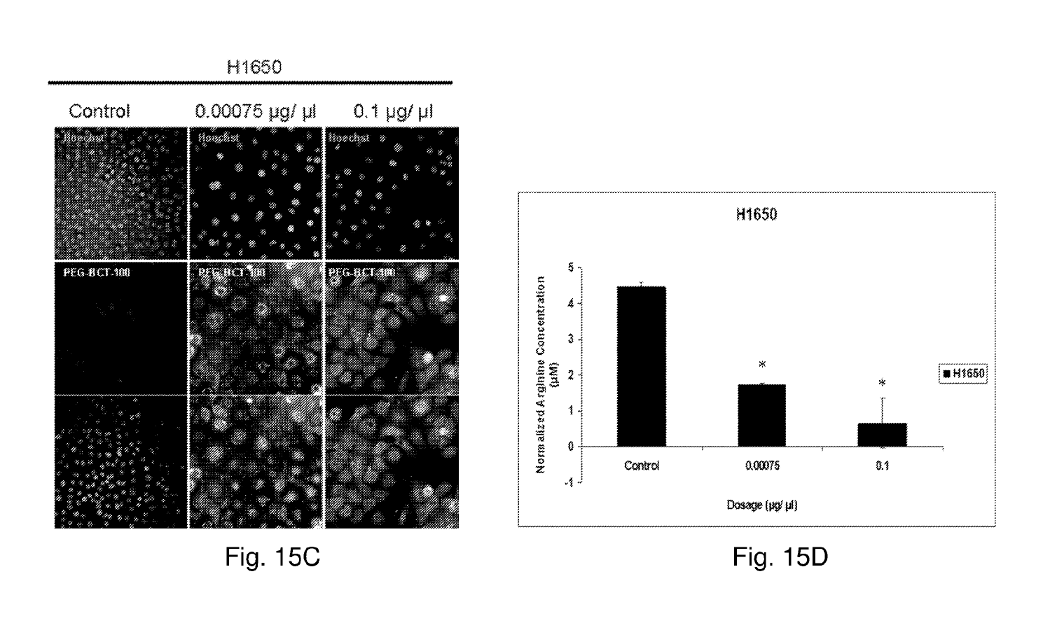

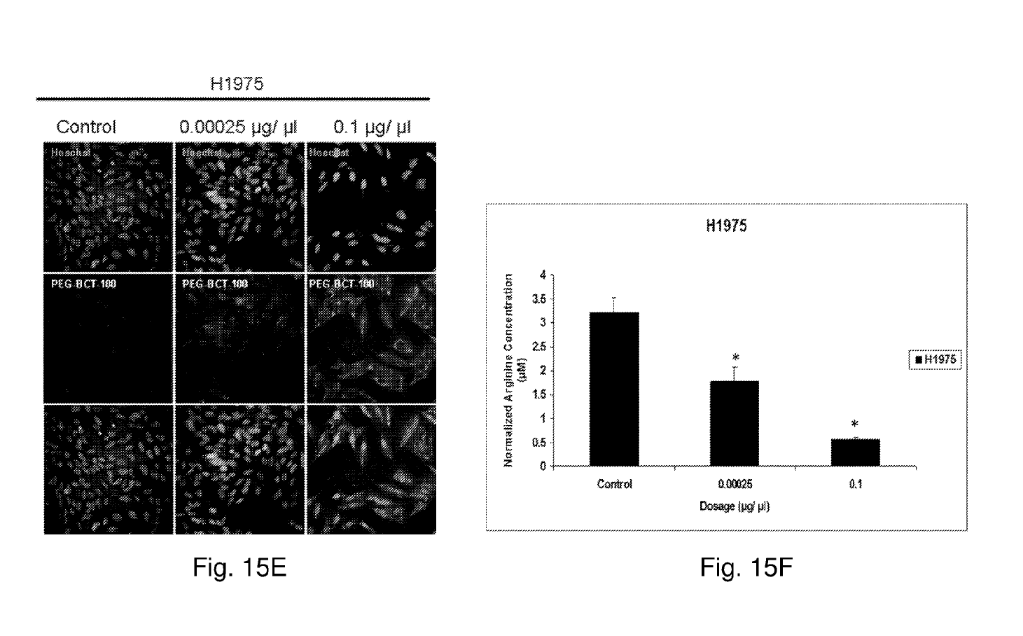

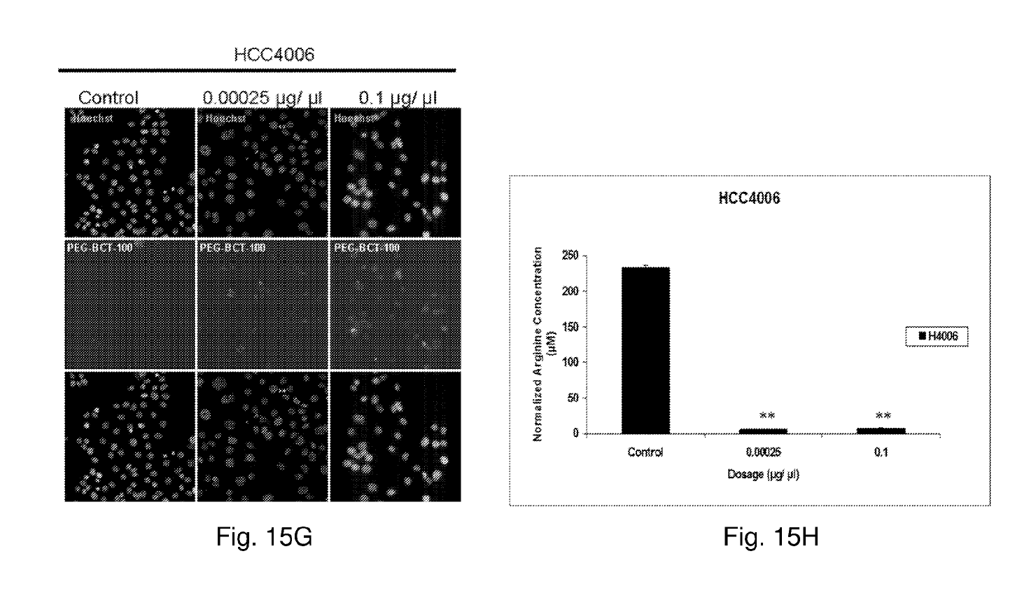

In one example embodiment, four lung adenocarcinoma cell lines (i.e. H358, H1650, H1975, and HCC4006), obtained from ATCC, are treated with PEG-BCT-100 at IC₅₀ concentrations and 0.1 μg/μl. Internalization of PEG-BCT-100 by lung adenocarcinoma cells are assessed by immunocytochemistry using anti-PEG-antibodies. Detection is done using anti-rabbit Alexa 488 conjugated secondary antibody and visualization is performed using a fluorescent microscope. The PEG-BCT-100 treated lung adenocarcinoma cells are lysed in RIPA buffer for determination of arginine level by K7733 arginine ELISA kit from Immunodiagnostik. Results of this study are presented in FIGS. 15A-15H.

FIGS. 15A-15H show that PEG-BCT-100 is able to penetrate the cells of the examined lung adenocarcinoma cell lines, as shown by the cytosolic staining of PEG-BCT-100 by anti-PEG antibody from Abcam and Alexa Fluor 488 conjugated goat anti-rabbit secondary antibody. FIGS. 15A-15H also show that PEG-BCT-100 depletes intracellular arginine significantly. (* p<0.05, ** p<0.01).

As illustrated in FIGS. 15A-15F, PEG-BCT-100 is able to penetrate into H1975, H1650, and HCC4006 cells at IC₅₀ level and is able to penetrate into all examined lung adenocarcinoma cells at 0.1 μg/μl. PEG-BCT-100 is able to deplete cytosolic arginine level significantly in all examined lung adenocarcinoma cell lines.

### Example 4

One example embodiment studies in vivo efficacy of PEG-BCT-100 in lung adenocarcinoma.

In one example embodiment, ten million lung adenocarcinoma cells from cell lines H1650, H1975, and HCC4006 are engrafted subcutaneously in BALB/cnude mice (4 weeks old with body weight of 10-14 g). Body weight, clinical signs and survival times are recorded.

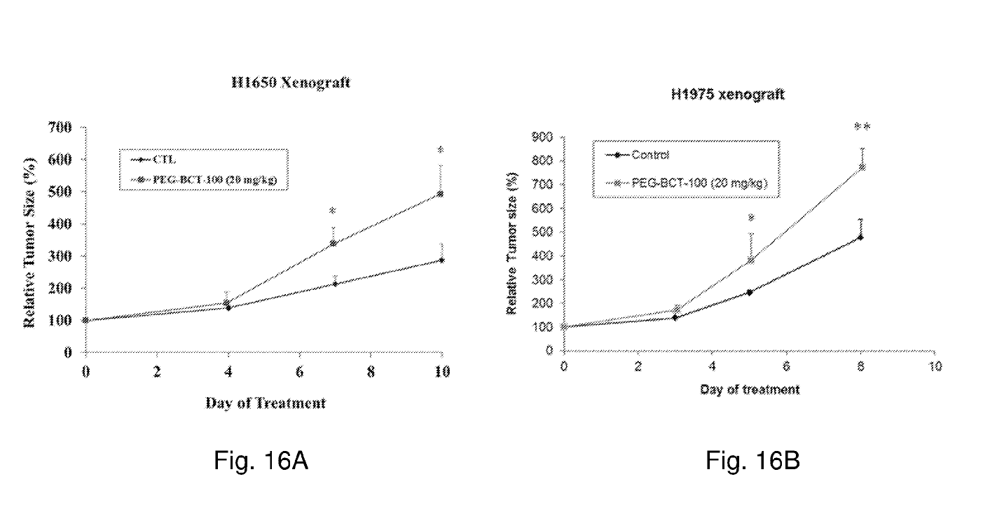

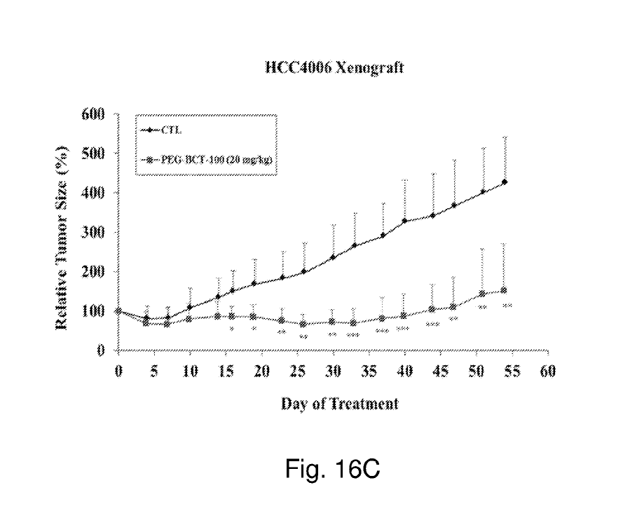

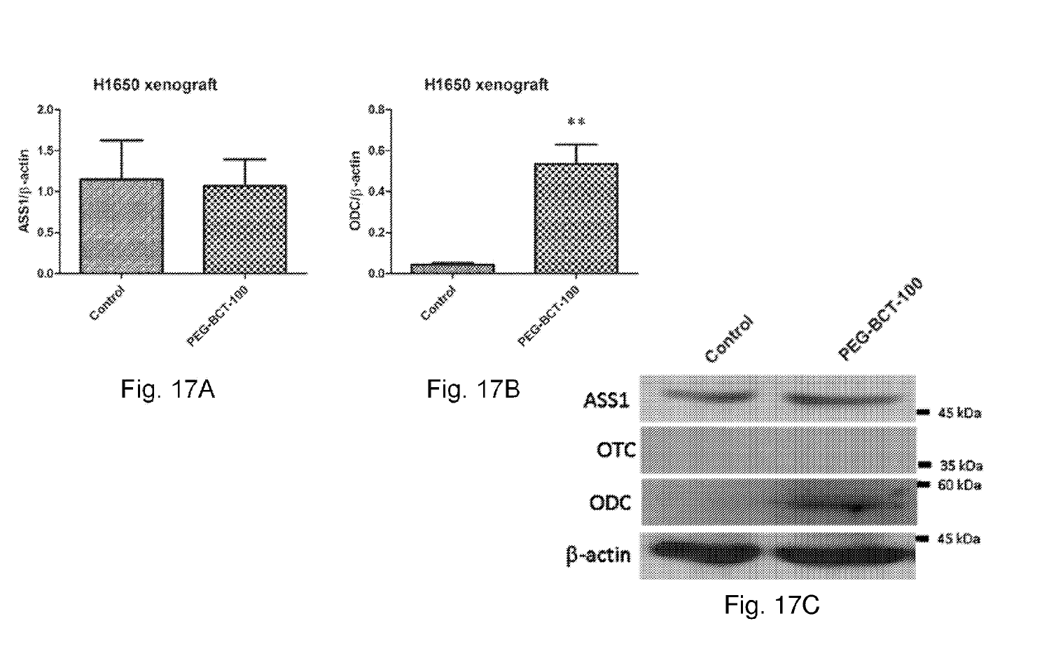

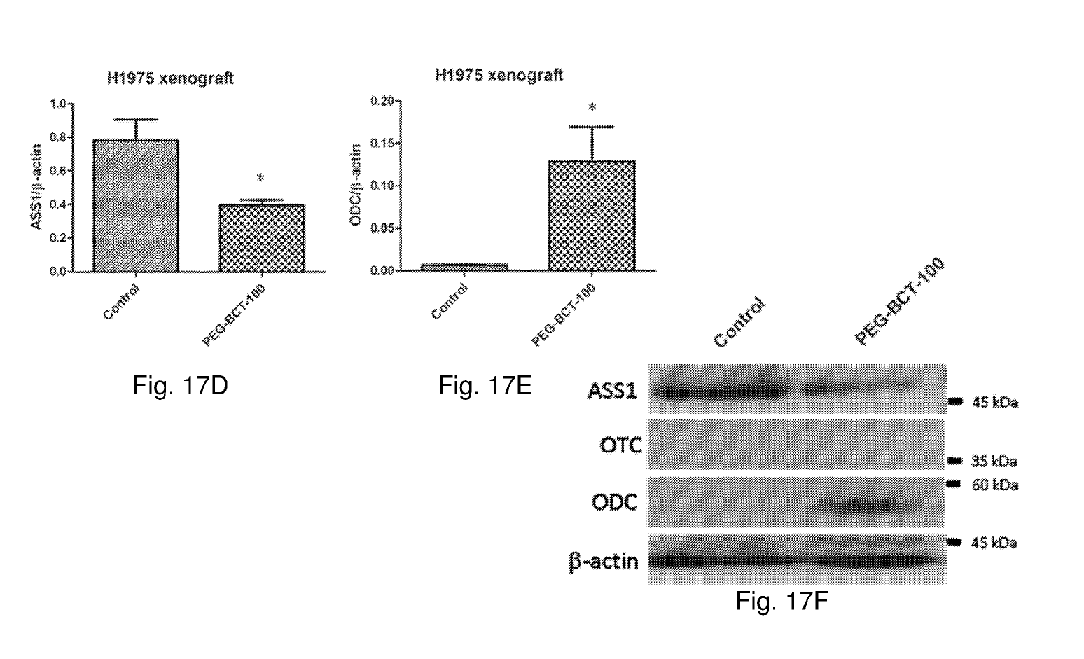

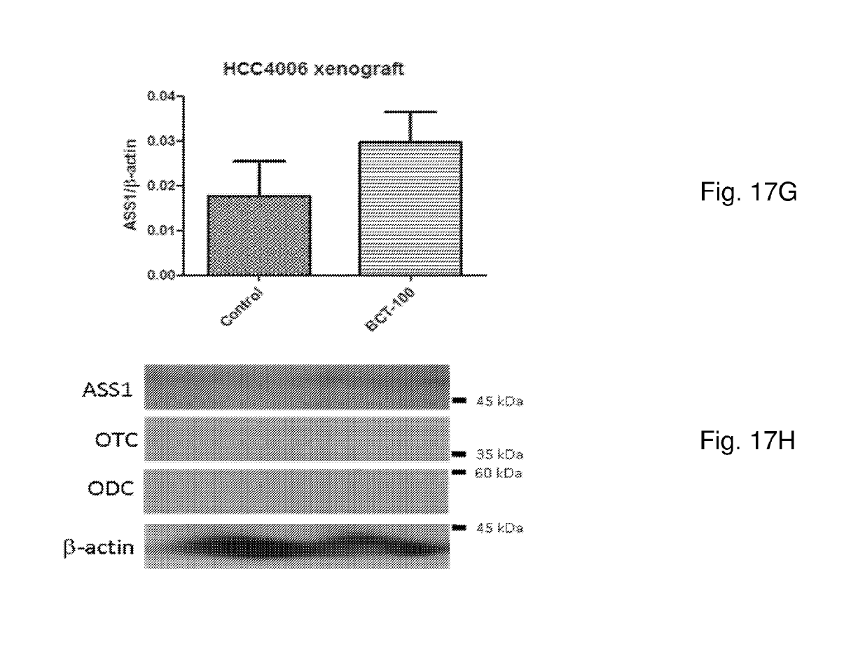

Two groups of the mice, with eight mice in each group, are tested. Control group (negative control) receives physiological saline and treatment group receives 20 mg/kg of PEG-BCT-100. PEG-BCT-100 is administered via intraperitoneal (IP) injection, twice weekly, until euthanization. Results of this study are presented in FIGS. 16A-17H.

FIGS. 16A-16C respectively show effects of PEG-BCT-100 on H1650, H1975, and HCC4006 lung adenocarcinoma xenografts in BALB/c nude mice. PEG-BCT-100 induces lung adenocarcinoma proliferation in xenograft models of H1975 and H1650 but suppresses tumor growth in HCC4006. (* p<0.05, ** p<0.01, *** p<0.001.)

FIGS. 17A-17H show in vivo ASS1, OTC and ODC expression in H1650, H1975, and HCC4006 tumor xenografts by Western blot in the control arm and the PEG-BCT-100 treatment arm. ASS1 expression decreases in H1975 xenograft model while remains unchanged in H1650 and HCC4006 xenograft models on comparing the control groups with the control group and the PEG-BCT-100 treated group. ODC expression increases in H1650 and H1975 xenograft models but remains negative in HCC4006 xenograft model on comparing the control arm and the PEG-BCT-100 treatment arm. (* p<0.05.)

As illustrated in FIGS. 16A-17H, PEG-BCT-100 at 20 mg/kg promotes tumour growth in H1650 and H1975 xenograft models, in which paradoxical growth stimulation is observed. PEG-BCT-100 at 20 mg/kg inhibits tumour growth in HCC4006 xenografts. In vivo ornithine decarboxylase (ODC) expression is analyzed for each xenograft, with or without PEG-BCT-100 treatment, which is correlated with tumour size. It is found that in H1650 and H1975 xenografts, but not HCC4006, ODC is over-expressed upon PEG-BCT-100 treatment. Thus, PEG-BCT-100, as a single agent, induces lung adenocarcinoma proliferation in selected xenograft models of H1975 and H1650, but suppresses tumor growth in HCC4006 xenograft.

### Example 5

One example embodiment studies in vivo efficacy of PEG-BCT-100 combined with α-Difluoromethylornithine (DFMO) in lung adenocarcinoma treatment.

In one example embodiment, ten million lung adenocarcinoma cells from cell lines H1650, H1975 and HCC4006 are engrafted subcutaneously in BALB/cnude mice (4 weeks old with body weight of 10-14 g). Body weight, clinical signs and survival times are recorded.

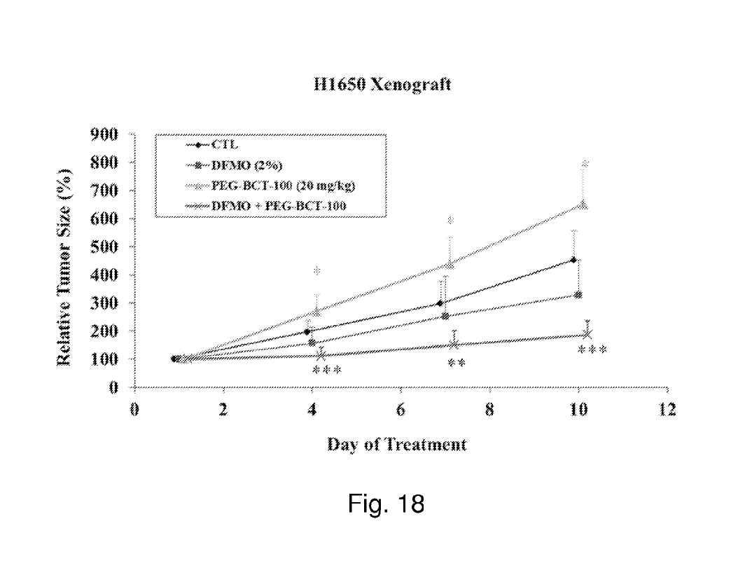

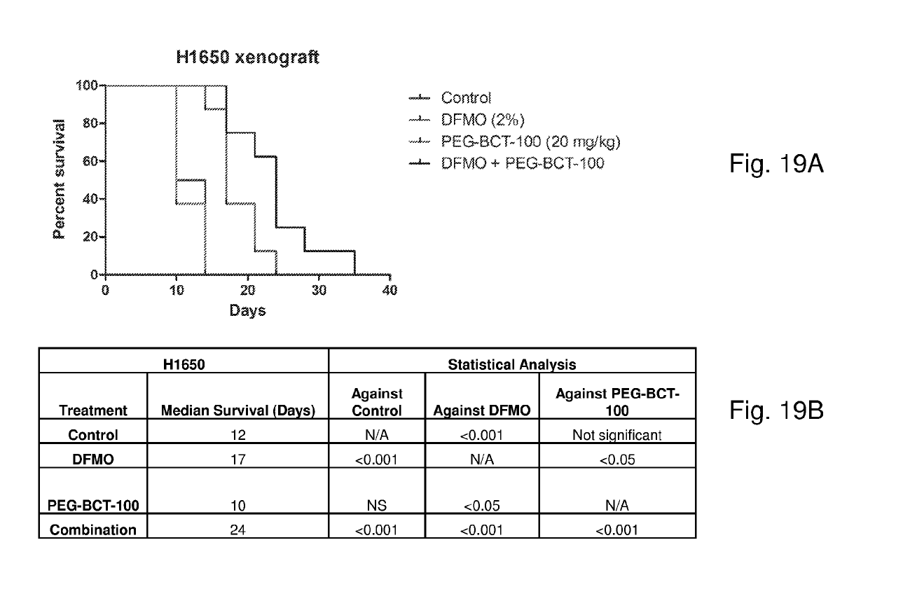

Four groups of the mice, with eight mice in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intraperitoneally, twice a week, until euthanization while DFMO is supplied in drinking water at 2% w/v. Results of this study are presented in FIGS. 18-22.

FIG. 18 shows in vivo effects of PEG-BCT-100 and/or DFMO in H1650 lung adenocarcinoma xenografts. (* p<0.05, ** p<0.01, *** p<0.001 for inter-group comparisons.)

FIGS. 19A-19B show median survival of BALB/c nude mice with H1650 lung adenocarcinoma xenografts upon PEG-BCT-100 and/or DFMO treatments. Combination treatment of PEG-BCT-100 and DFMO (2% w/v) shows significant improvement in median survival.

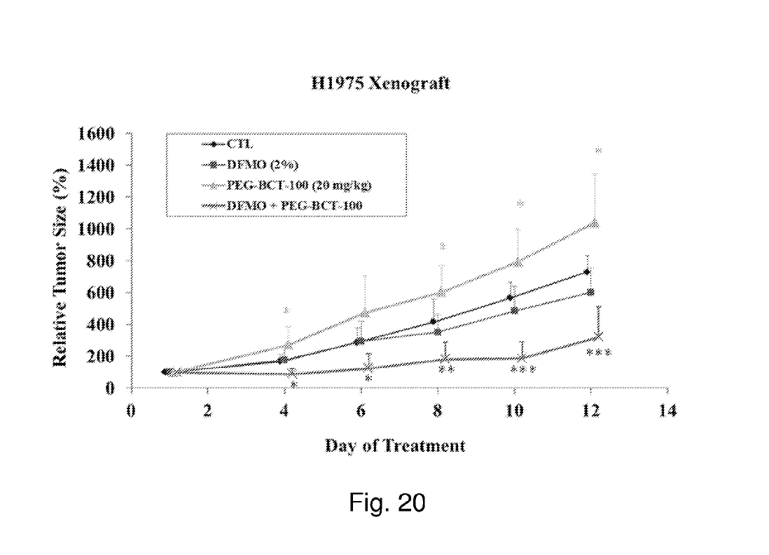

FIG. 20 shows in vivo effects of PEG-BCT-100 and/or DFMO in H1975 lung adenocarcinoma xenograft model. (* p<0.05, ** p<0.01, *** p<0.001 for inter-group comparisons.)

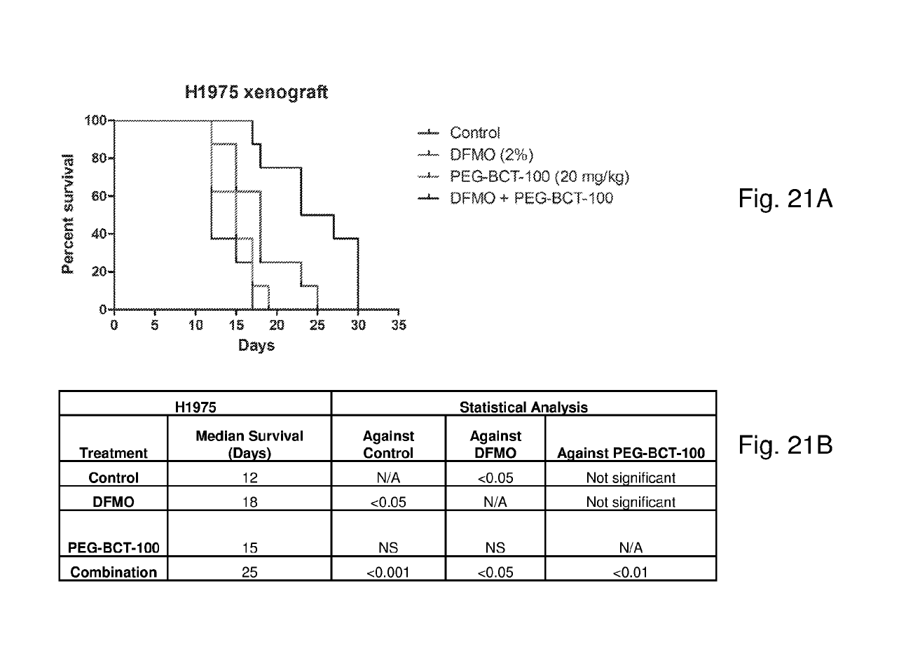

FIGS. 21A-21B show median survival of BALB/c nude mice with H1975 lung adenocarcinoma xenografts treated with PEG-BCT-100 and/or DFMO. Combination treatment of PEG-BCT-100 and DFMO (2% w/v) shows significant improvement in median survival.

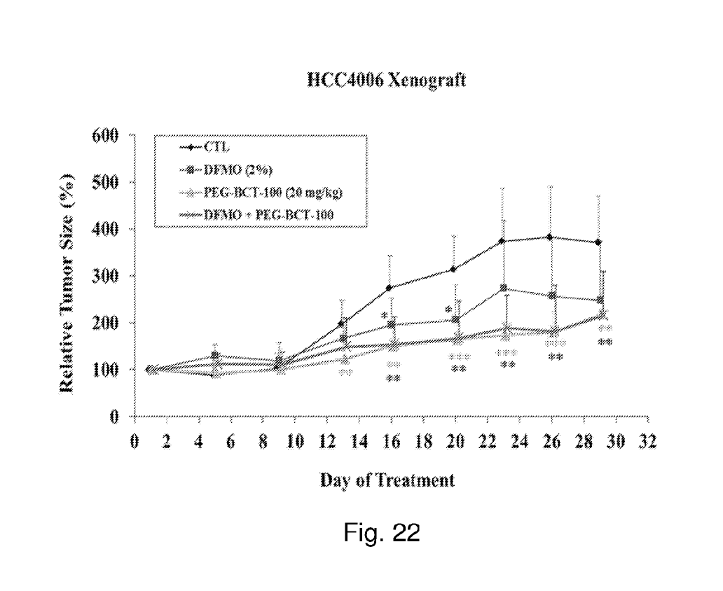

FIG. 22 shows in vivo effects of PEG-BCT-100 and/or DFMO in HCC4006 lung adenocarcinoma xenograft model.

In H1650 xenograft, as shown in FIG. 18, the combination of PEG-BCT-100 and DFMO decreases the rate of tumor growth as compared with the tumor growth rate for DFMO or the tumor growth rate for PEG-BCT100. As further illustrated in FIG. 19B, the median survival for the combination treatment group (24 days) is longer than either the DFMO group (17 days) or PEG-BCT-100 group (10 days). Thus, the results show that PEG-BCT-100 in combination with DFMO presents a synergistic effect in suppressing the growth of tumor in H1650 cell line.

In H1975 xenograft, as shown in FIG. 20, the combination of PEG-BCT-100 and DFMO decreases the rate of tumor growth as compared with the tumor growth rate for DFMO or the tumor growth rate for PEG-BCT-100. As further illustrated in FIG. 21B, the median survival for the combination treatment group (25 days) is longer than either the DFMO group (18 days) or PEG-BCT-100 group (15 days). Thus, the results show that PEG-BCT-100 in combination with DFMO presents a synergistic effect in suppressing the growth of tumor in H1975 cell line.

As illustrated in FIGS. 18-22, PEG-BCT-100 at 20 mg/kg promotes tumour growth in H1650 and H1975 xenograft models. DFMO is used as an ODC inhibitor. ODC is previously shown to be upregulated in H1650 and H1975 xenografts upon PEG-BCT-100 treatment, possibly leading to enhanced tumorigenesis. When both drugs are combined, the previously observed PEG-BCT-100-induced tumour growth is aborted, resulting in significant tumour shrinkage compared to control group. On the other hand, DFMO does not enhance the anti-tumour effect of PEG-BCT-100 in HCC4006 xenograft model, which does not show upregulation of ODC upon PEG-BCT-100 treatment alone.

In short, PEG-BCT-100, when combined with DFMO, produces a significant anti-tumor effect leading to prolonged survival in lung adenocarcinoma xenograft models.

### Example 6

One example embodiment studies in vivo efficacy of PEG-BCT-100 in SU-DHL-6 B cell lymphoma treatment.

In one example embodiment, the SU-DHL-6 human lymphoma cells are maintained in vitro as a suspension culture in RPMI1640 medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

One day before tumour inoculation, all mice are sub-lethally irradiated with 60Co (200 rad). Each mouse is inoculated subcutaneously at the right flank region with SU-DHL-6 tumour cells (5×10⁶) in 0.1 ml of PBS/Matrigel (1:1) for tumour development. The treatments start when the mean tumour size reaches 125 mm³. The date of tumour cell inoculation is denoted as day 0.

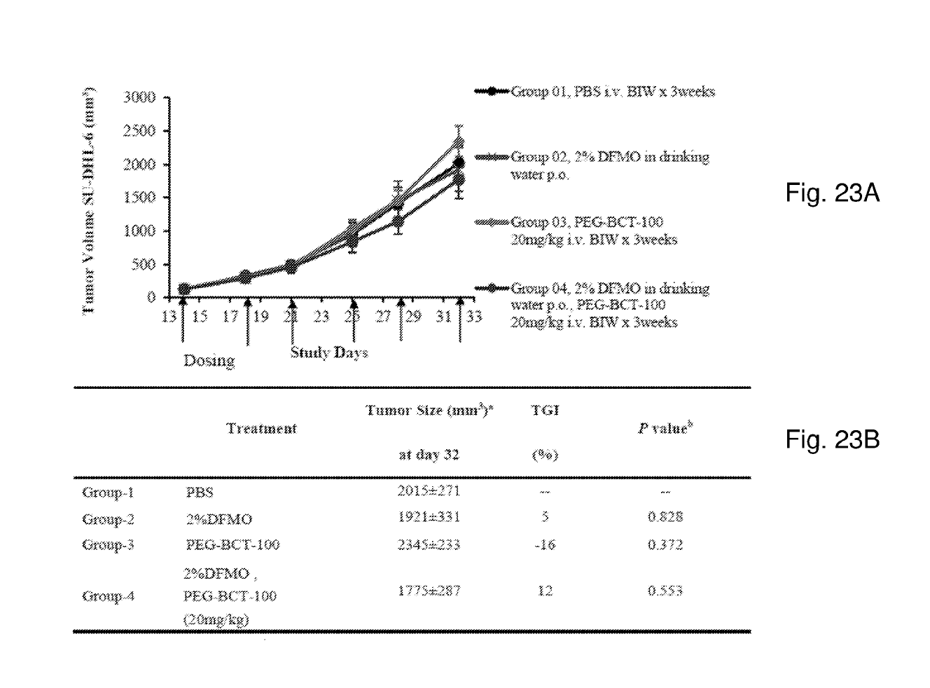

Four groups of animals, with eight animals in each group, are tested. Control group (negative control) receives physiological saline, and the three treatment groups respectively receive 20 mg/kg of PEG-BCT-100 alone, 2% w/v DFMO in drinking water, and a combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 23A-23B.

FIG. 23A shows in vivo effects of PEG-BCT-100 and/or DFMO in SU-DHL-6 B cell lymphoma xenograft model. FIG. 23B shows anti-tumour activity of PEG-BCT-100 and/or DFMO in SU-DHL-6 B cell lymphoma xenograft model.

As illustrated in FIGS. 23A-23B, 2% DFMO in combination with PEG-BCT-100 (20 mg/kg) shows some anti-tumor activity but without any significance (TGD=12%, p=0.553). Thus, PEG-BCT-100 and 2% DFMO, when used as a single agent or in combination, do not produce a significant anti-tumour effect in SU-DHL-6 B cell lymphoma cell line-derived xenograft model.

### Example 7

One example embodiment studies in vivo efficacy of PEG-BCT-100 in SU-DHL-10 B Cell lymphoma treatment.

In one example embodiment, SU-DHL-10 human lymphoma cells are maintained in vitro culture in RPMI1640 medium supplemented with 20% fetal bovine serum at 37° C. in an atmosphere of 5% CO₂ in air. The tumour cells are routinely sub-cultured twice weekly. The cells in an exponential growth phase are harvested and counted for tumour inoculation.

One day before tumour inoculation, all mice are sub-lethally irradiated with 60Co (200 rad). Each mouse is inoculated subcutaneously at the right flank region with SU-DHL-10 tumour cells (1×10⁷) in 0.1 ml of PBS/Matrigel (1:1) for tumour development. The treatments start when the mean tumour volume reached 116 mm³. The date of tumour cell inoculation is denoted as day 0.

Four treatment groups of animals, with eight animals in each group, are tested. Control group (negative control) receives physiological saline, 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v.

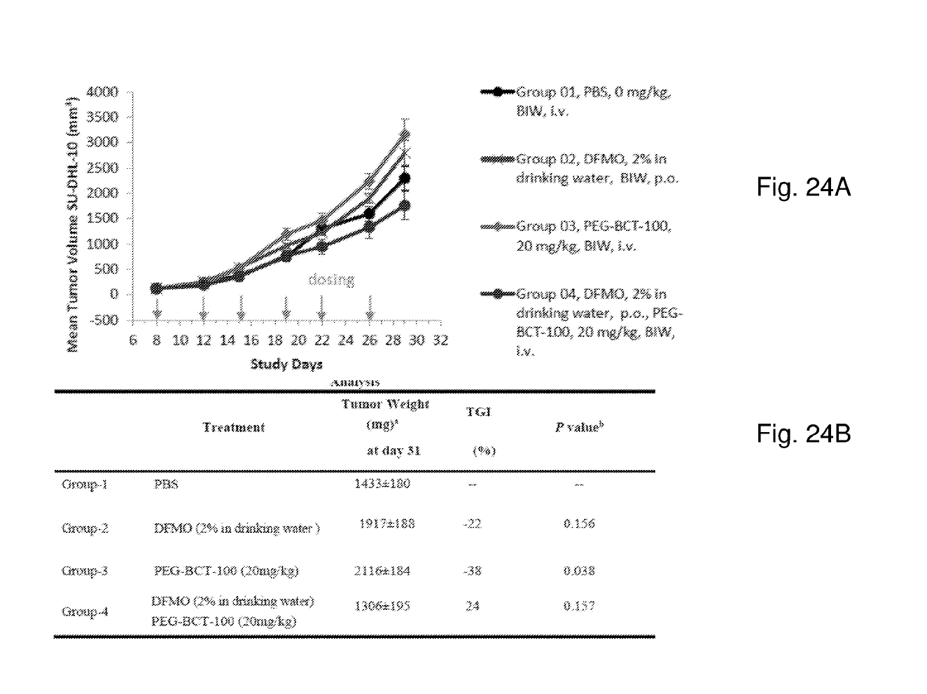

FIG. 24A shows in vivo effects of PEG-BCT-100 and/or DFMO in SU-DHL-10 B cell lymphoma xenograft model. FIG. 24B shows anti-tumour activity of PEG-BCT-100 and/or DFMO in SU-DHL-10 B cell lymphoma xenograft model.

As illustrated in FIGS. 24A-24B, PEG-BCT-100 at 20 mg/kg slightly promotes SU-DHL-10 tumour growth. DFMO, on the other hand, shows no significant anti-tumour effect either being used singly or in combination with PEG-BCT-100. Thus, PEG-BCT-100 and 2% DFMO, when used in combination, do not produce a significant anti-tumour effect in SU-DHL-10 B cell lymphoma cell line-derived xenograft model.

### Example 8

One example embodiment studies in vivo efficacy of PEG-BCT-100 in Kasumi-1 leukemia treatment.

In one example embodiment, the Kasumi-1 tumour cells are maintained in vitro as a suspension culture in RPMI1640 medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

All mice are γ-irradiated (200 rad) for 24 h before tumour cell injection. Kasumi-1 tumour cells (1×10⁷) in 0.2 ml of PBS are mixed with cultrex in a 1:1 ratio. Each mouse is inoculated subcutaneously at the right flank region with the tumour cells suspension for tumour development. The treatments start when the mean tumour size reached 114 mm³. The date of tumour cell inoculation is denoted as day 0.

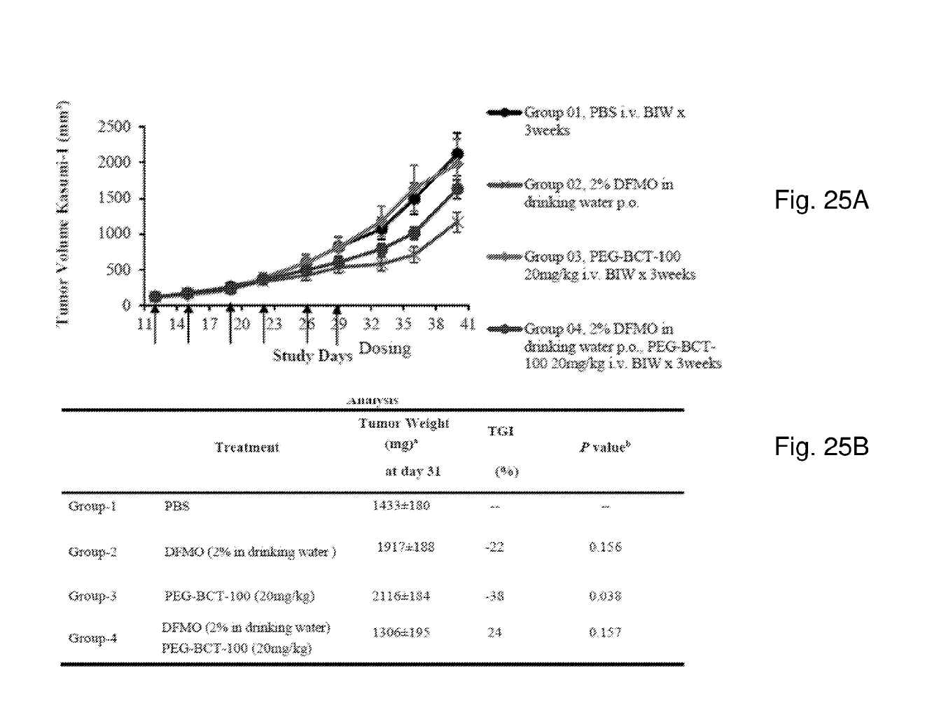

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 25A-25B.

FIG. 25A shows in vivo effects of PEG-BCT-100 and/or DFMO in Kasumi-1 leukemia xenograft model. FIG. 25B shows anti-tumour activity of PEG-BCT-100 and/or DFMO in Kasumi-lleukemia xenograft model.

As illustrated in FIGS. 25A-25B, 2% DFMO in drinking water shows significant anti-tumour activity (TGI=45%, p=0.013). On the other hand, PEG-BCT-100 at 20 mg/kg shows no significant anti-tumour effect when applied as a single agent or in combination with DFMO. Thus, PEG-BCT-100 and 2% DFMO, when used in combination, do not produce a significant anti-tumour effect in Kasumi-1 leukemia cell line-derived xenograft model.

### Example 9

One example embodiment studies In vivo efficacy of PEG-BCT-100 in K562 leukemia treatment.

In one example embodiment, the K-562 tumour cells are maintained in vitro as a suspension culture in RPMI1640 medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

Each mouse is inoculated subcutaneously at the right flank region with K-562 tumour cells (5×10⁶) in 0.1 ml of PBS/Matrigel (1:1) for tumour development. The treatments start when the mean tumour size reaches 125 mm³. The date of tumour cell inoculation is denoted as day 0.

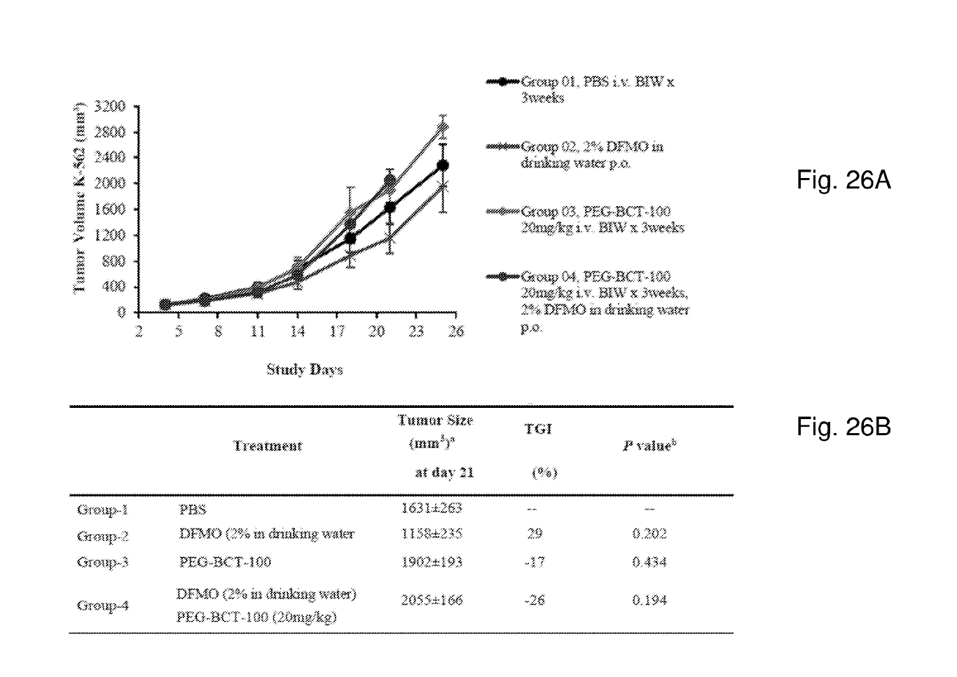

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 26A-26B.

FIG. 26A shows in vivo effects of PEG-BCT-100 and/or DFMO in K-562 leukemia xenograft model. FIG. 26B shows anti-tumour activity of PEG-BCT-100 and/or DFMO in K-562 leukemia xenograft model.

As illustrated in FIGS. 26A-26B, PEG-BCT-100 and/or DFMO show no significant anti-tumour effect. Thus, PEG-BCT-100 and/or 2% DFMO, when used as single agents or in combination, do not produce a significant anti-tumour effect in K-562 leukemia cell line-derived xenograft model.

### Example 10

One example embodiment studies in vivo efficacy of PEG-BCT-100 in Hep G2 hepatocellular carcinoma treatment.

In one example embodiment, the Hep G2 human liver cancer cells are maintained in vitro culture in RPMI1640 medium supplemented with 10% fetal bovine serum at 37° C. in an atmosphere of 5% CO₂ in air. The tumour cells are routinely sub-cultured twice weekly. The cells in an exponential growth phase are harvested and counted for tumour inoculation.

Each mouse is inoculated subcutaneously at the right flank region with Hep G2 tumour cells (1×10⁷) in 0.2 ml of PBS (1:1 Matrigel) for tumour development. The treatments start when the mean tumour volume reaches 124 mm³. The date of tumour cell inoculation is denoted as day 0.

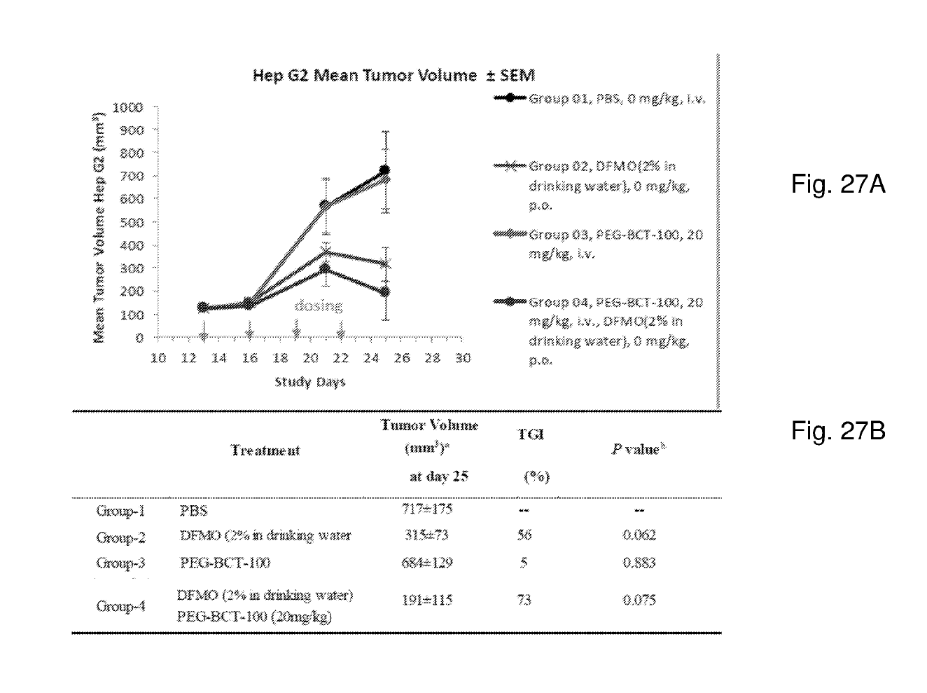

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 27A-27B.

FIG. 27A shows in vivo effects of PEG-BCT-100 and/or DFMO in Hep G2 hepatocellular carcinoma xenograft model. FIG. 27B shows anti-tumour activity of PEG-BCT-100 and/or DFMO in Hep G2 hepatocellular carcinoma xenograft model.

As illustrated in FIGS. 27A-27B, DFMO, when used as a single agent or in combination with PEG-BCT-100, shows a significant anti-tumour effect. However, the combination treatment of DFMO and PEG-BCT-100 is not significantly better than the DFMO single agent treatment. Thus, the major anti-tumour effect seen in the combination treatment of DFMO and PEG-BCT-100 is mainly due to DFMO as the combination treatment does not yield a significantly superior anti-tumour effect than DFMO single agent treatment in Hep G2 xenograft model.

### Example 11

One example embodiment studies in vivo efficacy of PEG-BCT-100 in Hep 3B hepatocellular carcinoma treatment.

In one example embodiment, the Hep 3B tumour cells are maintained in vitro culture in EMEM medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

Each mouse is inoculated subcutaneously at the right flank region with Hep 3B tumour cells (5×10⁶) in 0.1 ml of PBS (1:1 Matrigel) for tumour development. The treatments start when the mean tumour size reaches 119 mm³. The date of tumour cell inoculation is denoted as day 0.

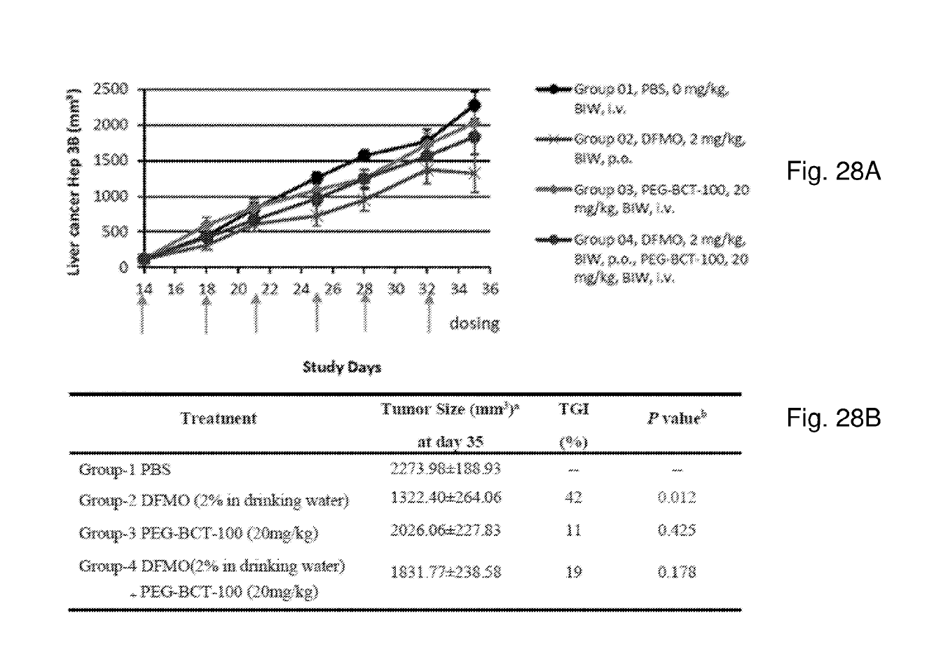

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 28A-28B.

FIG. 28A shows in vivo effects of PEG-BCT-100 and/or DFMO in Hep 3B hepatocellular carcinoma xenograft model. FIG. 28B shows anti-tumour activity of PEG-BCT-100 and/or DFMO in Hep 3B hepatocellular carcinoma xenograft model.

As illustrated in FIGS. 28A-28B, DFMO, when used as a single agent, shows a significant anti-tumour effect. PEG-BCT-100, when used as a single agent or in combination with DFMO, does not produce a significant anti-tumour effect. Thus, in comparison to PEG-BCT-100, DFMO could potentially be a better treatment reagent against Hep3B hepatocellular carcinoma.

### Example 12

One example embodiment studies in vivo efficacy of PEG-BCT-100 in MIA-PaCa-2 pancreatic cancer.

In one example embodiment, the MIA-PaCa-2 tumour cells are maintained in vitro culture in EMEM medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

Each mouse is inoculated subcutaneously at the right flank region with MIA-PaCa-2 tumour cells (5×10⁶) in 0.1 ml of PBS (1:1 Matrigel) for tumour development. The treatments start when the mean tumour size reaches 124 mm³. The date of tumour cell inoculation was denoted as day 0.

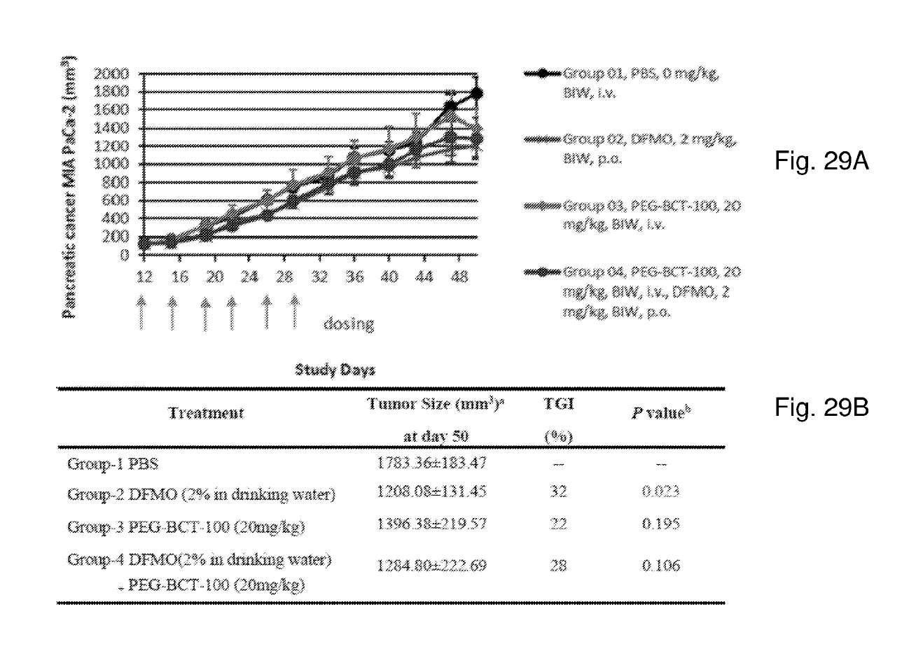

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 29A-29B.

FIG. 29A shows in vivo effects of PEG-BCT-100 and/or DFMO in MIA-PaCa-2 pancreatic cancer xenograft model. FIG. 29B shows anti-tumour activity of PEG-BCT-100 and/or DFMO in MIA-PaCa-2 pancreatic cancer xenograft model.

As illustrated in FIGS. 29A-29B, DFMO, when used as a single agent, shows a significant anti-tumour effect. PEG-BCT-100, when used as a single agent or in combination with DFMO, do not produce a significant anti-tumour effect. Thus, in comparison to PEG-BCT-100, DFMO could potentially be a better treatment reagent against MIA-PaCa-2 pancreatic cancer.

### Example 13

One example embodiment studies in vivo efficacy of PEG-BCT-100 in CAPAN-1 pancreatic cancer.

In one example embodiment, the CAPAN-1 tumour cells are maintained in vitro culture in EMEM medium supplemented with 10% heat inactivated fetal bovine serum at 37° C. in an atmosphere of 5% CO₂ in air. The tumour cells are routinely sub-cultured twice weekly. The cells growing in an exponential growth phase are harvested and counted for tumour inoculation.

Each mouse is inoculated subcutaneously at the right flank region with CAPAN-1 tumour cells (5×10⁶) in 0.1 ml of PBS (1:1 Matrigel) for tumour development. The treatments start when the mean tumour size reaches 119 mm³. The date of tumour cell inoculation is denoted as day 0.

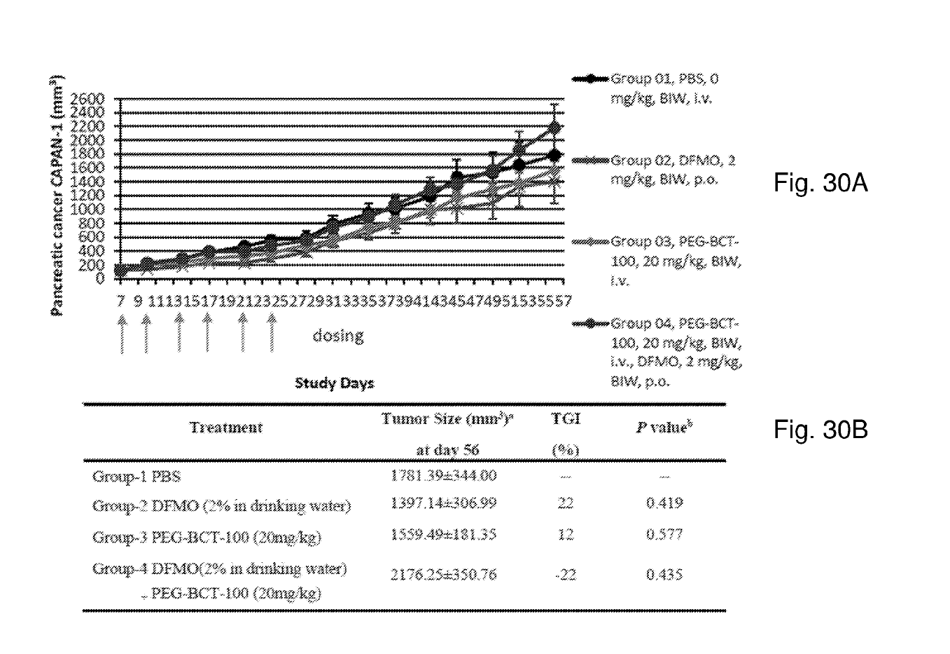

Four treatment groups of animals, with eight animals in each group, are tested. Control group (negative control) receives physiological saline, and the three treatment groups respectively receive 20 mg/kg of PEG-BCT-100 alone, 2% w/v DFMO in drinking water, and combination of 20 mg/kg of PEG-BCT-100 and 2% w/v DFMO. PEG-BCT-100 is administered intravenously, twice a week, until euthanization while DFMO is supplied in drinking water at 2% w/v. Results of this study are presented in FIGS. 30A-30B.

FIG. 30A shows in vivo effects of PEG-BCT-100 and/or DFMO in CAPAN-1 pancreatic cancer xenograft model. FIG. 30B shows anti-tumour activity of PEG-BCT-100 and/or DFMO in MIA-PACA-2 pancreatic cancer xenograft model.

As illustrated in FIGS. 30A-30B, PEG-BCT-100 and/or DFMO show no significant anti-tumour effect. Thus, PEG-BCT-100 and/or 2% DFMO, when used as single agents or in combination, do not produce a significant anti-tumour effect in CAPAN-1 pancreatic cancer cell line-derived xenograft model.

As used herein, the term “arginine reducing compound” or “arginine depleting compound” means any compound that reduces arginine or depletes arginine. Examples include, but are not limited to, arginase or its analogs.

As used herein, the term “ornithine decarboxylase (ODC) inhibitor” or “ODC blocking agent” means any compound that inhibits or blocks ODC. Examples include, but are not limited to, DFMO or its analogs.

As used herein, the term “pegylated arginase”, “pegylated human arginase”, or “pegylated recombinant human arginase” refers to an arginase of the present invention modified by pegylation to increase the stability of the enzyme and minimize immunoreactivity.

In an example embodiment, the arginase is a recombinant human arginase I that has an amino acid sequence of SEQ ID NO:1 and a nucleic acid sequence of SEQ ID NO:2. In one example embodiment, the pegylated arginase has at least one polyethylene glycol (PEG) molecule that covalently links with an amino acid residue or with more than one amino acid residue of the arginase. By way of example, at least one PEG molecule covalently links with a lysine residue or with more than one lysine residues of the arginase. In another example embodiment, the PEG has a molecular weight of 5 KDa.

In one example embodiment, the pegylation of the arginase is achieved by covalently conjugating a PEG molecule with the arginase using a coupling agent. Examples of a coupling agent includes, but are not limited to, methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-succinimidyl glutarate (mPEG-SG), mPEG-N-hydroxyl-succinimide (mPEG-NHS), mPEG-tresylate, and mPEG-aldehyde. By way of example, the coupling agent is methoxy polyethylene glycol-succinimidyl propionate 5000 with an average molecular weight of 5K.

In an example embodiment, the pegylated recombinant human arginase, PEG-BCT-100, disclosed in this application includes a recombinant human arginase I that has an amino acid sequence of SEQ ID NO.1 and a nucleic acid sequence of SEQ ID NO.2, in which the recombinant human arginase I has at least one PEG molecule that covalently links with an amino acid residue or with more than one amino acid residue of the recombinant human arginase I. In one example embodiment, the recombinant human arginase I has about 6-12 PEG molecules per arginase. By way of example, the PEG molecule covalently links with a lysine residue or with more than one lysine residues of the recombinant human arginase I.

In another example embodiment, the pegylated recombinant human arginase, PEG-BCT-100, disclosed in this application includes a recombinant human arginase I that has an amino acid sequence of SEQ ID NO.3 and a nucleic acid sequence of SEQ ID NO.4, in which the recombinant human arginase I has six additional histidines at an amino-terminal end thereof, and at least one PEG molecule that covalently links with an amino acid residue or with more than one amino acid residue of the recombinant human arginase I. In an example embodiment, the six histidines are added for ease of purification. In one example embodiment, the recombinant human arginase I has about 6-12 PEG molecules per arginase. By way of example, the PEG molecule covalently links with a lysine residue or with more than one lysine residues of the recombinant human arginase I.

As used herein, the terms “combination therapy”, “combined treatment” or “in combination” means any form of concurrent or parallel treatment with at least two distinct therapeutic agents.

As used herein, the term “subject” means any mammal having cancer that requires treatment, includes but is not limited to human.

As used herein, the term “therapeutically effective amount” means the amount of the arginine reducing compound and/or the ornithine decarboxylase (ODC) inhibitor to be effective in treating cancer cells/disease of a particular type. A specific “therapeutically effective amount” will vary according to the particular condition being treated, the physical condition and clinical history of the subject, the duration of the treatment, and the nature of the combination of agents applied and its specific formulation.

As used herein, the term “synergistic” and its various grammatical variations means an interaction between the arginine reducing compound and the ODC inhibitor wherein an observed effect (e.g., cytotoxicity) in the presence of the drugs together is higher than the sum of the individual effects (e.g., cytotoxicities) of each drug administered separately. In one embodiment, the observed combined effect of the drugs is significantly higher than the sum of the individual effects.

The compounds or compositions of the present invention may be administered to a subject by a variety of routes, for example, orally, intrarectally or parenterally (i.e. subcutaneously, intravenously, intramuscularly, intraperitoneally, or intratracheally).

As used herein, the term “DFMO” means eflornithine or α-difluoromethylornithine.

As used herein, the term “ODC negative” means a cell is unable to express the enzyme, ornithine decarboxylase, either genotypically or phenotypically.

As used herein, the term “ODC positive” means a cell is able to express the enzyme, ornithine decarboxylase, either genotypically or phenotypically.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference to describe and disclose specific information for which the reference was cited in connection with.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings.

What is claimed is:

1. A method of treating lung carcinoma in a subject in need thereof, comprising:
administering to the subject a therapeutically effective amount of an arginine reducing compound and a therapeutically effective amount of an ornithine decarboxylase (ODC) inhibitor to provide a combination therapy that has a synergistic therapeutic effect compared to an effect of the arginine reducing compound and an effect of the ODC inhibitor each administered alone.

2. The method of claim 1, wherein the lung carcinoma is lung adenocarcinoma.

3. The method of claim 1, wherein the arginine reducing compound is a pegylated recombinant human arginase.

4. The method of claim 1, wherein the arginine reducing compound is a pegylated recombinant human arginase, and the recombinant human arginase has an amino acid sequence of SEQ ID NO:1.

5. The method of claim 1, wherein the ODC inhibitor is difluoromethylornithine (DFMO).

6. The method of claim 1, wherein the arginine reducing compound and the ODC inhibitor are administered concurrently.

7. The method of claim 1, wherein cancer cells of the lung carcinoma are ODC positive.

8. The method of claim 1, wherein cancer cells of the lung carcinoma are ODC negative.

9. The method of claim 1, wherein cancer cells of the lung carcinoma are argininosuccinate synthase negative (ASS1⁻) or ornithine transcarbamylase negative (OTC⁻).

10. A method of blocking ornithine decarboxylase (ODC) in cancer cells in treating lung cancer in a subject in need thereof, the method comprising:
administering to the subject a therapeutically effective amount of an arginine depleting compound and a therapeutically effective amount of an ODC blocking agent, wherein administration of the arginase depleting compound and the ODC blocking agent provides a synergistic therapeutic effect compared to an effect in treating lung cancer of the arginine depleting compound and an effect in treating lung cancer of the ODC blocking agent each administered alone.

11. The method of claim 10, wherein the lung cancer is lung adenocarcinoma.

12. The method of claim 10, wherein the arginine depleting compound is a pegylated recombinant human arginase.

13. The method of claim 10, wherein the arginine depleting compound is a pegylated recombinant human arginase, and the recombinant human arginase has an amino acid sequence of SEQ ID NO:1.

14. The method of claim 10, wherein the ODC blocking agent is difluoromethylornithine (DFMO).

15. The method of claim 10, wherein the cancer cells are ODC positive.

16. The method of claim 10, wherein the cancer cells are ODC negative.

17. The method of claim 10, wherein the cancer cells are argininosuccinate synthase negative or ornithine transcarbamylase negative.

18. A method for inhibiting proliferation of lung adenocarcinoma cancer cells, comprising:
contacting the lung adenocarcinoma cancer cells with an arginine depleting compound in combination with an ornithine decarboxylase (ODC) inhibitor;
wherein the combination provides a synergistic anti-cancerous effect compared to an effect of the arginine depleting compound and an effect of the ODC inhibitor each administered alone.

19. The method of claim 18, wherein the arginine depleting compound is a pegylated recombinant human arginase.

20. The method of claim 18, wherein the arginine depleting compound is a pegylated recombinant human arginase, and the recombinant human arginase has an amino acid sequence of SEQ ID NO:1.

21. The method of claim 18, wherein the ODC inhibitor is difluoromethylornithine (DFMO).

22. The method of claim 18, wherein the arginine depleting compound and the ODC inhibitor are administered simultaneously.

23. A pharmaceutical composition for use in a synergistic treatment of lung cancer, the pharmaceutical composition comprising:
an arginine depleting compound; and
an inhibitor of ornithine decarboxylase (ODC).

24. The pharmaceutical composition of claim 23, wherein the lung cancer is lung adenocarcinoma.

25. The pharmaceutical composition of claim 23, wherein the arginine depleting compound is a pegylated recombinant human arginase.

26. The pharmaceutical composition of claim 23, wherein the arginine depleting compound is a pegylated recombinant human arginase, and the recombinant human arginase has an amino acid sequence of SEQ ID NO:1.

27. The pharmaceutical composition of claim 23, wherein the inhibitor of ODC is difluoromethylornithine (DFMO).

28. The pharmaceutical composition of claim 23, wherein an amount of the arginine depleting compound and an amount of the inhibitor of ODC are effective for therapy in a subject, and the subject is a human.
